(19) United States
(12) Patent Application Publication
(10) Pub. No.: US 2012/0178089 A1
(43) Pub. Date: Jul. 12, 2012

(54) MATERIALS AND METHODS FOR THE IDENTIFICATION OF DRUG-RESISTANT CANCERS AND TREATMENT OF SAME

(76) Inventors: Fenghuang Zhan, Salt Lake City, UT (US); Maurizio Zangari, Salt Lake City, UT (US); Guido Tricot, North Liberty, IA (US)

(21) Appl. No.: 13/335,283

(22) Filed: Dec. 22, 2011

Related U.S. Application Data
(63) Continuation-in-part of application No. PCT/US2010/039927, filed on Jun. 25, 2010.
(60) Provisional application No. 61/269,661, filed on Jun. 26, 2009.

(51) Int. Cl. C12Q 1/68; G01N 33/566

(52) U.S. Cl. 435/6.12; 436/501; 435/7.1; 435/7.92

(57) ABSTRACT

Disclosed herein are diagnostic methods for identifying cancer and predicting drug resistance. The assays involve the detection of NEK2 gene expression alone or in combination with other genes or clinical factors. The test is suitable for diagnosing and monitoring treatment of subjects having or suspected of having a neoplastic disease, such as multiple myeloma. The disclosure also relates to inhibitors of NEK2 for the treatment of cancer, including drug-resistant multiple myeloma.

### CROSS-REFERENCE TO RELATED APPLICATIONS

This application is a continuation-in-part of PCT International Patent Application No. PCT/US2010/039927, filed Jun. 25, 2010, which claims priority to U.S. Provisional Patent Application No. 61/269,661, filed Jun. 26, 2009, the contents of which are hereby incorporated by reference in their entireties.

### STATEMENT OF GOVERNMENT SUPPORT

This invention was made with United States government support under Grant #CA115399 awarded by the National Institutes of Health. The United States government has certain rights in the invention.

### TECHNICAL FIELD

The present disclosure relates generally to the diagnosis, prognosis, and management of disease, including cancer. In particular, the present technology relates to methods for detecting gene expression alterations associated with cancer. The present disclosure also relates to inhibitors of NEK2 for the treatment of cancer, including drug-resistant multiple myeloma.

### BACKGROUND

The following discussion of the background is merely provided to aid the reader in understanding the invention and is not admitted to describe or constitute prior art.

Multiple myeloma (MM) is an incurable disease with survival rates that range from a few months to more than 15 years. It is estimated that patients with myeloma have 10¹²-10¹³ myeloma cells and at complete remission may still have up to 10⁹ myeloma cells present. Since tumor burden does not appear to be the major prognostic marker in multiple myeloma, this suggests that achieving a prolonged event free survival is not dependent upon the absolute reduction in myeloma burden, but rather on genetic characteristics of myeloma cells. In fact, in many different types of malignancies, achievement of a complete remission and eradication of all macroscopic disease has not resulted in the improvement of overall survival rates.

As the majority of tumor cells can be killed by conventional chemotherapies and cancer cells affected by those therapies have a limited proliferative potential, it may be that drug resistant cancer cells have the characteristics of stem cell quiescence and self-renewal, and, as such, would be more resistant to chemotherapeutics than tumor cells with limited proliferative potential. These genetic characteristics may be present at diagnosis in a small sub-fraction, but masked by the chemo-sensitive cells, or, alternatively may be a consequence of the chemotherapy itself. Identification of those particular drug-resistant cancers at the time of a diagnosis would help an oncologist make the right treatment decision for a patient.

### SUMMARY

The present disclosure is based on the discovery that the expression of NEK2 may be detected in patient samples and that such expression can have clinical value in the diagnosis and prognosis of certain disease states. In one aspect, the present disclosure provides a method for determining a diagnosis or prognosis of a neoplastic disease in a subject, the method comprising detecting the expression of one or more of NEK2, BIRC5, TOP2A, PHF19, EZH2, NUSAP1, NDC80. CDKN3, RHOC, and PTPRC in a test sample from the subject, wherein an elevation of one or more of NEK2, BIRC5, TOP2A, PHF19, EZH2, NUSAP1, NDC80. CDKN3, RHOC, and PTPRC expression in the subject compared to a reference level is an indication of a diagnosis of a neoplastic disease or an increased likelihood of recurrence of a neoplastic disease. In one embodiment, an increase of NEK2, BIRC5, TOP2A, PHF19, EZH2, NUSAP1, NDC80. CDKN3, RHOC, and PTPRC expression in the subject compared to the reference level is an indication of drug-resistant multiple myeloma.

In another aspect, the present disclosure provides a method for determining a diagnosis or prognosis of a neoplastic disease in a subject, the method comprising detecting the expression of NEK2 in a test sample from the subject, wherein an elevation of NEK2 expression in the subject compared to a reference level is an indication of a diagnosis of a neoplastic disease or an increased likelihood of recurrence of a neoplastic disease. In one embodiment, the sample is a body fluid sample or a biopsy sample.

In one embodiment, an increase of NEK2 expression in the subject compared to the reference level is an indication of drug-resistant cancer. In one embodiment, the neoplastic disease is multiple myeloma. In one embodiment, an increase of NEK2 expression in the subject compared to the reference level is an indication of drug-resistant multiple myeloma. In one embodiment, a difference in the level of NEK2 expression in the subject compared to a reference level is an indication of early multiple myeloma recurrence and decreased overall survival in a subject with multiple myeloma.

In one embodiment, a difference in the level of NEK2 expression in the subject compared to a reference level is an indication of a diagnosis of small cell lung carcinoma, breast cancer, glioma, adult acute myeloid leukemia, bladder cancer, mantel cell lymphoma, or mesothelioma in the subject. In one embodiment, the difference in the level of NEK2 expression is an increase of NEK2 expression in the subject compared to the reference level. In one embodiment, the reference level is the level in a comparable sample from one or more healthy individuals.

In one embodiment, the detecting comprises amplifying a fragment of the NEK2 mRNA. In one embodiment, the amplifying is accomplished by polymerase chain reaction (PCR). In one embodiment, the detecting comprises RT-PCR. In one embodiment, the amplifying employs a detectably labeled primer. In one embodiment, the detecting is accomplished using the TaqMan® PCR detection system.

In one embodiment, the detecting comprises measuring the presence, absence, or amount of a NEK2 protein in the sample. In one embodiment, the measuring uses an antibody that specifically binds to an NEK2 protein. In one embodiment, the measuring is by an ELISA assay, a Western blot assay, or an immunohistochemical assay.

In another aspect, the present disclosure provides a method for treating myeloma, lung or breast cancer in a subject, the method comprising administering to the subject an effective amount of the kinase inhibitor KP372-1. In one embodiment, cancer is multiple myeloma. In one embodiment, the cancer is drug-resistant multiple myeloma. In one embodiment, the multiple myeloma is resistant to proteasome inhibitors. In one embodiment, the proteasome inhibitor is selected from the group consisting of: bortezomib, Disulfiram, Salinosporamide A, Carfilzomib, CEP-18770 and MLN9708.

### BRIEF DESCRIPTION OF THE FIGURES

FIGS. 1a to 1e are graphs of Kaplan-Meier analysis showing high NEK2 expression is linked to a poor prognosis in myeloma. FIG. 1f is a graph showing box plots of NEK2 gene expression (y axis) in plasma cells from 22 healthy individuals (NPC), 44 patients with MGUS, 12 patients with SMM, 351 newly diagnosed MM patients in TT2, 214 newly diagnosed MM patients in TT3, 51 patients with relapsed myeloma in TT2, and 9 myeloma cell lines (MMCL). FIG. 1g and FIG. 1h are graphs of NEK2 expression in myeloma cells after chemo-therapy or relapsed myelomas, respectively.

FIG. 2a is a series of western blots showing increased NEK2 expression in cancer cells ARP1, KMS28PE, OCI-MY5, H1299, and MCF7, and normal fibroblast cells BJ transfected with NEK2-cDNA. FIGS. 2b-2g are a series of graphs showing that over-expression of NEK2 in cancer cells and normal cells induced cell proliferation. FIG. 2h is a graph showing NEK2 mRNA expression detected by real-time PCR was significantly higher in drug-resistant RPMI8226, ARP1, and MCF7 cells compared to their parental cancer cell lines, respectively (** P<0.01). FIG. 2i are western blots showing the protein levels of NEK2 were significantly higher in drug-resistant cancer cells compared to their parental control cells.

FIGS. 2j-21 are graphs showing that over-expression of NEK2 in cancer cells induces drug resistance to bortezomib (Velcade), doxorubicin, and etoposide.

FIGS. 3a and 3b present data from real-time PCR and western blots, respectively, showing NEK2 expression was significantly inhibited in NEK2-shRNA cells compared to the scramble (SCR)-transfected control cells. FIGS. 3c and 3d show NEK2-shRNAs induced ARP1 cell apoptosis and growth inhibition. FIG. 3e is a graph showing NEK snRNA-induced cell growth inhibition was partially abrogated by NEK2-cDNA over-expression.

FIGS. 4a-4g are a series of graphs showing NEK2 high-expression is linked to a poor prognosis in various cancers.

FIGS. 5a-5h are a series of graphs showing high expression of NEK2 in tumor tissues. NEK2-expression in cancer tissues (TT) compared to their relative normal tissues (NT), including head and neck squamous cell carcinoma (FIG. 5a), bladder carcinoma (FIG. 5b), glioblastoma (FIG. 5c), T-cell acute lymphoblastic leukemia (FIG. 5d), colon carcinoma (FIG. 5e), hepatocellular carcinoma (FIG. 5f), melanoma (FIG. 5g) and ovarian adenocarcinoma (FIG. 5h). FIG. 5i is a graph showing NEK2-expression in pulmonary carcinoids. FIG. 5j is a graph showing high NEK2-expression in advanced lung adenocarcinoma specimens determined by TNM stages (T1-T4).

FIGS. 6a-6h are a series of graphs showing the effects of silencing NEK2 on cancer cell growth and survival. NEK2-shRNAs significantly induced cancer cell growth inhibition (a, c, e, g) and cell death (b, d, f, h).

FIG. 7a is a graph showing the results of screening a Kinase Inhibitor Library. FIG. 7b is a series of western blots showing that the compound of NI-2 inhibits NEK2 expression in cancer cells over-expressing NEK2.

FIGS. 8a-8f are a series of graphs showing the NEK2 inhibitor KP372-1 induced cell growth inhibition and death in cancer cells with over-expressed NEK2.

FIGS. 9a-9d are a series of graphs showing the treatment with KP372-1 overcomes drug-resistance in cancer cells. The myeloma cell lines KMS28PE (A & B) and ARP1 (C & D) including both bortezomib-sensitive and bortezomib-resistant (vel-R) were treated with KP372-1 at concentrations of 1 μM and 5 μM for 5 days.

FIG. 10a is a chart showing the survival curve of mice following tumor injection. FIG. 10b is a graph of tumor volumes of mice following tumor injection. The treatments of KP372-1 and bortezomib as well as the combination of KP372-1 and bortezomib reduced tumor burden (P<0.05).

### DETAILED DESCRIPTION

The present disclosure relates inter alia to the diagnosis of cancer and the identification of cancers that are drug-resistant in patients, particularly methods for the quantification of mRNA or protein levels of one or more biomarkers in order to assist an oncologist in the determination of an appropriate chemotherapeutic regime for a patient. The biomarkers may be selected from the group consisting of: NEK2, BIRC5, TOP2A, PHF19, EZH2, NUSAP1, NDC80. CDKN3, RHOC, and PTPRC. The present disclosure also relates to detection methods and kits that utilize proteins, nucleic acid molecules and antibodies for e.g., diagnosis, determination of the stage of cancer and monitoring the treatment of cancer. The disclosure also relates to inhibitors of NEK2 for the treatment of cancer, including drug-resistant multiple myeloma.

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a nucleic acid” includes a combination of two or more nucleic acids, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent or drug to a subject or subject includes any route of introducing or delivering to a subject a compound to perform its intended function. Administration can be carried out by any suitable route, including orally, intranasally, parenterally (intravenously, intramuscularly, intraperitoneally, or subcutaneously), rectally, or topically. Administration includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the terms “amplification” or “amplify” mean one or more methods known in the art for copying a target nucleic acid, e.g., NEK2 mRNA, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp. 13-20; Wharam et al., Nucleic Acids Res., 2001, 29(11):E54-E54; Hafner et al., Biotechniques 2001, 30(4):852-6, 858, 860; Zhong et al., Biotechniques, 2001, 30(4):852-6, 858, 860.

The terms “determining,” “measuring,” “assessing,” and “assaying” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present, as well as determining whether it is present or absent.

As used herein, the term “effective amount” or “pharmaceutically effective amount” or “therapeutically effective amount” of a composition, is a quantity sufficient to achieve a desired therapeutic and/or prophylactic effect, e.g., an amount which results in the prevention of, or a decrease in, the symptoms associated with a disease that is being treated. The amount of a composition of the invention administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present invention can also be administered in combination with one or more additional therapeutic compounds.

The term “clinical factors” as used herein, refers to any data that a medical practitioner may consider in determining a diagnosis or prognosis of disease. Such factors include, but are not limited to, the patient's medical history, a physical examination of the patient, complete blood count, examination of blood cells or bone marrow cells, cytogenetics, and immunophenotyping of blood cells.

The term “comparable” or “corresponding” in the context of comparing two or more samples, means that the same type of sample (e.g., whole blood) is used in the comparison. For example, an expression level of NEK2 mRNA or protein in a sample of whole blood can be compared to an expression level of NEK2 in another whole blood sample. In some embodiments, comparable samples may be obtained from the same individual at different times. In other embodiments, comparable samples may be obtained from different individuals (e.g., a patient and a healthy individual). In general, comparable samples are normalized by a common factor. For example, body fluid samples are typically normalized by volume body fluid and cell-containing samples are normalized by protein content or cell count.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical art for a particular disease or disorder, e.g., a neoplastic disease.

The term “enzyme linked immunosorbent assay” (ELISA) as used herein refers to an antibody-based assay in which detection of the antigen of interest is accomplished via an enzymatic reaction producing a detectable signal. An ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generate a detectable product when contacted by appropriate substrates.

As used herein, a “fragment” in the context of a nucleic acid refers to a sequence of nucleotide residues which hare at least about 5 nucleotides, at least about 7 nucleotides, at least about 9 nucleotides, at least about 11, nucleotides, or at least about 17, nucleotides. A fragment is typically less than about 300 nucleotides, less than about 100 nucleotides, less than about 75 nucleotides less than about 50 nucleotides, or less than about 30 nucleotides. In certain embodiments, the fragments can be used in polymerase chain reaction (PCR), or various hybridization procedures to identify or amplify identical or related DNA molecules.

The term “neoplastic diseases” as used herein refers to cancers of any kind and origin and precursor stages thereof. Accordingly, the term “neoplastic disease” includes the subject matter identified by the terms “neoplasia”, “neoplasm”, “cancer”, “pre-cancer” or “tumor”. A neoplastic disease is generally manifest by abnormal cell division resulting in an abnormal level of a particular cell population. The abnormal cell division underlying a neoplastic disease is typically inherent in the cells and not a normal physiological response to infection or inflammation. In some embodiments, neoplastic diseases for diagnosis using methods provided herein include carcinoma. By “carcinoma,” it is meant a benign or malignant epithelial tumor and includes, but is not limited to, hepatocellular carcinoma, breast carcinoma, prostate carcinoma, non-small cell lung carcinoma, colon carcinoma, CNS carcinoma, melanoma, ovarian carcinoma, or renal carcinoma. An exemplary neoplastic disease includes, but is not limited to, multiple myeloma.

As used herein, “nucleic acid” refers broadly to segments of a chromosome, segments or portions of DNA, cDNA, and/or RNA. Nucleic acid may be derived or obtained from an originally isolated nucleic acid sample from any source (e.g., isolated from, purified from, amplified from, cloned from, or reverse transcribed from sample DNA or RNA).

As used herein, the term “oligonucleotide” refers to a short polymer composed of deoxyribonucleotides, ribonucleotides or any combination thereof. Oligonucleotides are generally between about 10 and about 100 nucleotides in length. Oligonucleotides are typically 15 to 70 nucleotides long, with 20 to 26 nucleotides being the most common. An oligonucleotide may be used as a primer or as a probe. An oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, or at least 95% sequence identity.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, a positive prognosis is one where a patient has a 50% probability of being cured of a particular cancer after treatment, while the average patient with the same cancer has only a 25% probability of being cured.

As used herein, the term “reference level” refers to a level of a substance which may be of interest for comparative purposes. In one embodiment, a reference level may be the expression level of a protein or nucleic acid expressed as an average of the level of the expression level of a protein or nucleic acid from samples taken from a control population of healthy (disease-free) subjects. In another embodiment, the reference level may be the level in the same subject at a different time, e.g., before the present assay, such as the level determined prior to the subject developing the disease or prior to initiating therapy. In general, samples are normalized by a common factor. For example, body fluid samples are normalized by volume body fluid and cell-containing samples are normalized by protein content or cell count.

As used herein, the term “time to recurrence” or “TTR” is used herein to refer to time in years to first recurrence censoring for second primary cancer as a first event or death without evidence of recurrence.

As used herein, the term “overall survival” or “OS” is used to refer to time in years from surgery to death from any cause. The calculation of this measure may vary depending on the definition of events to be either censored or not considered.

As used herein, the term “sample” or “test sample” refers to any liquid or solid material containing nucleic acids or proteins. In suitable embodiments, a test sample is obtained from a biological source (i.e., a “biological sample”), such as cells in culture or a tissue sample from an animal, most preferably, a human. In an exemplary embodiment, the sample is a tumor sample.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a neoplastic disease.

As used herein, “target nucleic acid” refers to segments of a chromosome, a complete gene with or without intergenic sequence, segments or portions a gene with or without intergenic sequence, or sequence of nucleic acids to which probes or primers are designed. Target nucleic acids may be derived from genomic DNA, cDNA, or RNA. As used herein, target nucleic acid may be native DNA or a PCR-amplified product. In one embodiment, the target nucleic acid is a fragment of a chromosome to be analyzed for methylation, e.g., a promoter region of a gene. In some embodiments, the target nucleic acid is a segment of the NEK2, BIRC5, TOP2A, PHF19, EZH2, NUSAP1, NDC80. CDKN3, RHOC, or PTPRC mRNA.

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for a disorder if, after receiving a therapeutic agent according to the methods of the present invention, the subject shows observable and/or measurable reduction in or absence of one or more signs and symptoms of a particular disease or condition.

The phrase “substantially the same as” in reference to a comparison of one value to another value for the purposes of clinical management of a disease or disorder means that the values are statistically not different. Differences between the values can vary, for example, one value may be within 20%, within 10%, or within 5% of the other value.

### Overview

Disclosed herein are methods for detecting the presence or absence of neoplastic diseases in subjects based, at least in part, on results of testing methods of the present technology on a sample. Further disclosed herein are methods for monitoring the status of subjects diagnosed with neoplastic diseases based at least partially on results of tests on a sample. The test samples disclosed herein are represented by, but not limited in anyway to, sputum, blood (or a fraction of blood such as plasma, serum, or particular cell fractions), lymph, mucus, tears, saliva, urine, semen, ascites fluid, whole blood, and biopsy samples of body tissue. This disclosure relates to methods of diagnosing and monitoring neoplastic diseases using the mRNA or protein expression level of a biomarker, e.g., NEK2, BIRC5, TOP2A, PHF19, EZH2, NUSAP1, NDC80. CDKN3, RHOC, and PTPRC.

Drug resistance is an important cause of treatment failure in cancer. Relapse phenotypes may be acquired via therapy-induced selection of resistant minor clones present at diagnosis rather than direct adaptation of the original disease. The present inventors discovered that over-expression of certain biomarkers promotes drug resistance, anti-apoptosis and cell proliferation in multiple myeloma. For example, NEK2, a member of the NIMA-related serine/threonine kinase family, has several putative roles in cell division, most notably in spindle formation and chromosome segregation. NEK2 expression is low in normal plasma cells and MGUS, but it increases its expression in about 20% newly diagnosed myelomas, 70% of relapsed myelomas, and in almost all myeloma cell lines. In other embodiments, the biomarker is selected from the group consisting of: BIRC5, TOP2A, PHF19, EZH2, NUSAP1, NDC80. CDKN3, RHOC, and PTPRC.

The present inventors also discovered that NEK2 is over-expressed in a number of different cancers. Regardless of the tissue of origin, all tumors have significantly increased NEK2 expression. It was further found that NEK2 expression level is proportionally increased from T1 to T2 and to T3 & T4 in the TNM stages in lung cancer. Comparing NEK2 expression in normal lung tissue, carcinoid lung tissue, adenocarcinoma, squamous cell carcinoma, and small cell lung carcinoma revealed that NEK2 is lowest in the normal tissue and highest in small cell lung carcinoma, which is known to be the most rapidly dividing lung tumor. Analysis of multiple primary cancer types including breast, lung, glioma, adult acute myeloid leukemia, bladder, mantle cell lymphoma, and mesothelioma cancer cells, showed over-expression of NEK2 which was associated with poor clinical outcome. As such, analysis of NEK2 expression is useful as a diagnostic tool to confirm and identify advanced stages and aggressive subtypes of cancers, such as transformed diffuse large B cell lymphoma from follicular lymphoma.

In one aspect, the methods generally provide for the detection, measuring, and comparison of a pattern of expression of one or more of NEK2, BIRC5, TOP2A, PHF19, EZH2, NUSAP1, NDC80. CDKN3, RHOC, or PTPRC protein or mRNA in a patient sample. Additional diagnostic markers may be combined with the profile to construct models for predicting the presence or absence or stage of a disease. For example, clinical factors of relevance to the diagnosis of neoplastic diseases, include, but are not limited to, the patient's medical history, a physical examination, complete blood count, and other markers. Moreover, biomarkers relevant to a particular neoplastic disease may be combined with a subject's NEK2, BIRC5, TOP2A, PHF19, EZH2, NUSAP1, NDC80. CDKN3, RHOC, or PTPRC expression profile to diagnose a disease or condition. In one embodiment, NEK2 expression is increased after chemotherapy and at disease relapse compared with diagnosis. In another embodiment, NEK2 expression is increased in drug resistant cancers.

Neoplastic diseases to which the methods of the present invention may include, for example, neoplastic lesions of the respiratory tract, of the urinary system, of the gastrointestinal tract of the anogenital tract, etc. Examples of cancer are cancer of the brain, breast, cervix, colon, head & neck, kidney, liver, lung, non-small cell lung, melanoma, mesothelioma, ovary, sarcoma, stomach, and uterus. In particular embodiments, the methods may be applied to the diagnosis or prognosis of multiple myeloma. Accordingly, the various aspects relate to the collection, preparation, separation, identification, characterization, and comparison of the abundance of the biomarker in a test sample. The technology further relates to detecting and/or monitoring a sample containing the biomarker protein or mRNA, which are useful, alone or in combination, to determine the presence or absence of a neoplastic disease or any progressive state thereof.

In one embodiment, the present methods are used to detect drug-resistant cancer in patients. Drug-resistant cancer is indicated by up-regulation (i.e. greater levels of mRNA or protein expression) of NEK2, BIRC5, TOP2A, PHF19, EZH2, NUSAP1, NDC80. CDKN3, RHOC, or PTPRC relative to expression levels seen in patients that do not have drug-resistant cancer. In a particular embodiments, the drug-resistant cancer is selected from the group consisting of pulmonary carcinoids lung carcinomas, adeno-squamous cell and small cell lung carcinomas, head and neck squamous cell carcinoma, bladder carcinoma, glioblastoma, T-cell acute lymphoblastic leukemia, colon carcinoma, hepatocellular carcinoma, melanoma, ovarian adenocarcinoma, breast cancer, glioma, adult acute myeloid leukemia, bladder cancer, mantle cell lymphoma, mesothelioma and lung adenocarcinoma.

In another embodiment, the methods of detecting drug-resistant cancer may be used to detect minimal residual disease, i.e. the presence of disease that was not eliminated through surgery and/or chemotherapy, in order to measure the effectiveness of chemotherapy. The methods may also be used for determining the stage of disease.

### Sample Collection and Preparation

The methods and compositions described herein may be used to detect nucleic acids associated with various genes using a biological sample obtained from an individual. The nucleic acid (DNA or RNA) may be isolated from the sample according to any methods well known to those of skill in the art. Biological samples may be obtained by standard procedures and may be used immediately or stored, under conditions appropriate for the type of biological sample, for later use.

Starting material for the detection assays is typically a clinical sample, which is suspected to contain the target nucleic acids, e.g., NEK2, BIRC5, TOP2A, PHF19, EZH2, NUSAP1, NDC80. CDKN3, RHOC, or PTPRC mRNA. An example of a clinical sample is a tissue from a tumor. Next, the nucleic acids may be separated from proteins and sugars present in the original sample. Any purification methods known in the art may be used in the context of the present invention. Nucleic acid sequences in the sample can successfully be amplified using in vitro amplification, such as PCR. Typically, any compounds that may inhibit polymerases are removed from the nucleic acids.

Methods of obtaining test samples are well known to those of skill in the art and include, but are not limited to, aspirations, tissue sections, swabs, drawing of blood or other fluids, surgical or needle biopsies, and the like. The test sample may be obtained from an individual or patient. The test sample may contain cells, tissues or fluid obtained from a patient suspected being afflicted with or cancer, e.g., multiple myeloma or non-small cell lung carcinoma. The test sample may be a cell-containing liquid or a tissue. Samples may include, but are not limited to, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples may also be processed, such as sectioning of tissues, fractionation, purification, or cellular organelle separation.

If necessary, the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of nucleic acid derived from the cells in the sample to detect using polymerase chain reaction.

### Nucleic Acid Extraction and Amplification

The nucleic acid to be amplified may be from a biological sample such as a tissue sample and the like. Various methods of extraction are suitable for isolating the DNA or RNA. Suitable methods include phenol and chloroform extraction. See Maniatis et al., Molecular Cloning, A Laboratory Manual, 2d, Cold Spring Harbor Laboratory Press, pp. 16-54 (1989). Numerous commercial kits also yield suitable DNA and RNA including, but not limited to, QLAamp™ mini blood kit, Agencourt Genfind™, Roche Cobas® Roche MagNA Pure® or phenol: chloroform extraction using Eppendorf Phase Lock Gels®, and the NucliSens extraction kit (Biomerieux, Marcy l'Etoile, France).

Nucleic acid extracted from cells or tissues can be amplified using nucleic acid amplification techniques well known in the art. By way of example, but not by way of limitation, these techniques can include the polymerase chain reaction (PCR), reverse transcriptase polymerase chain reaction (RT-PCR), nested PCR, ligase chain reaction. See Abravaya, K., et al., Nucleic Acids Research, 23:675-682, (1995), branched DNA signal amplification, Urdea, M. S., et al., AIDS, 7 (suppl 2):S11-S14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods, 35:273-286, (1991), Invader Technology, or other sequence replication assays or signal amplification assays may also be used. Some of these methods of amplification are described briefly below and are well-known in the art.

Some methods employ reverse transcription of RNA to cDNA. The method of reverse transcription and amplification may be performed by previously published or recommended procedures. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus thermophilus. For example, one method which may be used to convert RNA to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4:S83-S91, (1994).

In a suitable embodiment, PCR is used to amplify a target sequence of interest, e.g., NEK2, BIRC5, TOP2A, PHF19, EZH2, NUSAP1, NDC80. CDKN3, RHOC, or PTPRC. PCR is a technique for making many copies of a specific template DNA sequence. The reaction consists of multiple amplification cycles and is initiated using a pair of primer sequences that hybridize to the 5′ and 3′ ends of the sequence to be copied. The amplification cycle includes an initial denaturation, and typically up to 50 cycles of annealing, strand elongation and strand separation (denaturation). In each cycle of the reaction, the DNA sequence between the primers is copied. Primers can bind to the copied DNA as well as the original template sequence, so the total number of copies increases exponentially with time.

PCR can be performed as according to Whelan et al., J of Clin Micro, 33(3):556-561 (1995). Briefly, a PCR reaction mixture includes two specific primers, dNTPs, approximately 0.25 U of Taq polymerase, and 1×PCR Buffer.

The skilled artisan is capable of designing and preparing primers that are appropriate for amplifying a target or marker sequence. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill. For example, the length of a short nucleic acid or oligonucleotide can relate to its hybridization specificity or selectivity. Exemplary primers for detecting NEK2 mRNA may be designed based on the cDNA sequence available at GenBank Accession No. NM_{—}002497.

In some embodiments, the amplification may include a labeled primer or probe, thereby allowing detection of the amplification products corresponding to that primer or probe. In particular embodiments, the amplification may include a multiplicity of labeled primers or probes; such primers may be distinguishably labeled, allowing the simultaneous detection of multiple amplification products. In one embodiment, a primer or probe is labeled with a fluorogenic reporter dye that emits a detectable signal. While a suitable reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment, the detection reagent may be further labeled with a quencher dye such as Tamra, Dabcyl, or Black Hole Quencher® (BHQ), especially when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

Nucleic acids may be amplified prior to detection or may be detected directly during an amplification step (i.e., “real-time” methods). In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

In one embodiment, detection of a target nucleic acid, such as a nucleic acid from an NEK2 gene, is performed using the TaqMan® assay, which is also known as the 5′ nuclease assay (U.S. Pat. Nos. 5,210,015 and 5,538,848). The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced. During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target-containing template which is amplified during PCR.

In an illustrative embodiment, real time PCR is performed using TaqMan0 probes in combination with a suitable amplification/analyzer such as the ABI Prism® 7900HT Sequence Detection System. The ABI PRISM® 7900HT Sequence Detection System is a high-throughput real-time PCR system that detects and quantitates nucleic acid sequences. Real time detection on the ABI Prism 7900HT or 7900HT Sequence Detector monitors fluorescence and calculates Rn during each PCR cycle. The threshold cycle, or Ct value, is the cycle at which fluorescence intersects the threshold value. The threshold value is determined by the sequence detection system software or manually. The Ct can be correlated to the initial amount of nucleic acids or number of starting cells using a standard curve.

Other methods of probe hybridization detected in real time can be used for detecting amplification of a target or marker sequence flanking a tandem repeat region. For example, the commercially available MGB Eclipse™ probes (Epoch Biosciences), which do not rely on a probe degradation can be used. MGB Eclipse™ probes work by a hybridization-triggered fluorescence mechanism. MGB Eclipse™ probes have the Eclipse™ Dark Quencher and the MGB positioned at the 5′-end of the probe. The fluorophore is located on the 3′-end of the probe. When the probe is in solution and not hybridized, the three dimensional conformation brings the quencher into close proximity of the fluorophore, and the fluorescence is quenched. However, when the probe anneals to a target or marker sequence, the probe is unfolded, the quencher is moved from the fluorophore, and the resultant fluorescence can be detected.

Oligonucleotide probes can be designed which are between about 10 and about 100 nucleotides in length and hybridize to the amplified region. Oligonucleotides probes are preferably 12 to 70 nucleotides; more preferably 15-60 nucleotides in length; and most preferably 15-25 nucleotides in length. The probe may be labeled. Amplified fragments may be detected using standard gel electrophoresis methods. For example, in some embodiments, amplified fractions are separated on an agarose gel and stained with ethidium bromide by methods known in the art to detect amplified fragments.

### Protein Assays

In one aspect, the present invention provides methods of detecting a NEK2, BIRC5, TOP2A, PHF19, EZH2, NUSAP1, NDC80. CDKN3, RHOC, or PTPRC protein associated with a neoplastic disease, such as multiple myeloma. In one embodiment, the methods provide for detection of a NEK2 protein. The presence of NEK2 can be measured by immunoassay, using antibodies specific for NEK2 protein (GenBank Accession No. NP_{—}002488). Lack of antibody binding would indicate the absence of NEK2 protein molecules and suggest that the subject does not have or is not susceptible to a neoplastic disease associated with a overexpression of NEK2 protein. An exemplary NEK2 antibody is commercially available from BD Biosciences Pharmingen.

Antibodies which are specifically reactive with NEK2 protein may be obtained in a number of ways which will be readily apparent to those skilled in the art. The protein can be produced in a recombinant system using the nucleotide sequence of GenBank Accession No. NM_{—}002497. The recombinant protein can be injected into an animal as an immunogen to elicit polyclonal antibody production. The resultant polyclonal antisera may be used directly or may be purified by, for example, affinity absorption using recombinantly produced NEK2 coupled to an insoluble support.

In another alternative, monoclonal antibodies specifically immunoreactive with the mutant protein may be prepared according to well known methods (See, e.g., Kohler and Milstein, 1976, Eur. J. Immunol., 6:611), using a peptide as an immunogen, using it for selection or using it for both functions. These and other methods for preparing antibodies that are specifically immunoreactive with the recombinant protein of this invention are easily within the skill of the ordinary worker in the art.

In some embodiments, NEK2 proteins can be detected by immunohistochemistry, immunofluorescence, ELISPOT, ELISA, or RIA. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, immunohistochemistry, fluorescence microscopy, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with NEK2. The antibodies can be tested for their desired activity using the in vitro assays described herein, or by analogous methods.

By “specifically binds” is meant that an antibody recognizes and physically interacts with its cognate antigen (e.g., a NEK2 polypeptide) and does not significantly recognize and interact with other antigens; such an antibody may be a polyclonal antibody or a monoclonal antibody, which are generated by techniques that are well known in the art. The antibody can be bound to a substrate or labeled with a detectable moiety or both bound and labeled. The detectable moieties contemplated with the present compositions include fluorescent, enzymatic and radioactive markers.

### Diagnosis of Disease States

In certain embodiments, the level of NEK2, BIRC5, TOP2A, PHF19, EZH2, NUSAP1, NDC80. CDKN3, RHOC, or PTPRC protein or mRNA in a test sample from a patient is used in the diagnosis or prognosis of a neoplastic disease, such as multiple myeloma. In some embodiments, the level of biomarker protein or nucleic acid in a test sample is used to monitor response to therapy. The level of biomarker protein or mRNA before treatment can be compared to levels during treatment at regular intervals. A reduction in the biomarker proteins or mRNA provides an objective assessment of efficacy of therapy, and of patient compliance.

In some embodiments, the level of NEK2, BIRC5, TOP2A, PHF19, EZH2, NUSAP1, NDC80. CDKN3, RHOC, or PTPRC protein or mRNA in a test sample is used to diagnose a disease. The level biomarker protein or mRNA may be compared to a reference value to determine if the levels of the biomarkers are elevated or reduced relative to the reference value. Typically, the reference value is the level biomarkers measured in a comparable sample from one or more healthy individuals. An increase or decrease in the biomarkers may be used in conjunction with clinical factors other than the biomarkers to diagnose a disease.

Association between a pathological state (e.g., cancer and/or chemotherapy drug-resistance) and the aberration of a level of the biomarkers, e.g., NEK2, can be readily determined by comparative analysis in a normal population and an abnormal or affected population. Thus, for example, one can study the level of NEK2 protein or nucleic acid in both a normal population and a population affected with a particular pathological state. The study results can be compared and analyzed by statistical means. Any detected statistically significant difference in the two populations would indicate an association. For example, if the level of NEK2 protein or nucleic acid is statistically significantly higher in the affected population than in the normal population, then it can be reasonably concluded that higher level of NEK2 protein or nucleic acid is associated with the pathological state.

Statistical methods can be used to set thresholds for determining when the level of a biomarker in a subject can be considered to be different than or similar to a reference level. In addition, statistics can be used to determine the validity of the difference or similarity observed between a patient's level of the biomarker and the reference level. Useful statistical analysis methods are described in L.D. Fisher & G. vanBelle, Biostatistics: A Methodology for the Health Sciences (Wiley-Interscience, NY, 1993). For instance, confidence (“p”) values can be calculated using an unpaired 2-tailed t test, with a difference between groups deemed significant if the p value is less than or equal to 0.05. As used herein a “confidence interval” or “CI” refers to a measure of the precision of an estimated or calculated value. The interval represents the range of values, consistent with the data that is believed to encompass the “true” value with high probability (usually 95%). The confidence interval is expressed in the same units as the estimate or calculated value. Wider intervals indicate lower precision; narrow intervals indicate greater precision. Suitable confidence intervals of the invention are 90%, 95%, 97.5%, 98%, 99%, 99.5%, 99.9% and 99.99%. A “p-value” as used herein refers to a measure of probability that a difference between groups happened by chance. For example, a difference between two groups having a p-value of 0.01 (or p=0.01) means that there is a 1 in 100 chance the result occurred by chance. Suitable p values are 0.1, 0.05, 0.025, 0.02, 0.01, 0.005, 0.001, and 0.0001. Confidence intervals and p-values can be determined by methods well-known in the art. See, e.g., Dowdy and Wearden, Statistics for Research, John Wiley & Sons, New York, 1983.

Once an association is established between a level of the biomarker and a pathological state, then the particular physiological state can be diagnosed or detected by determining whether a patient has the particular aberration, i.e. elevated or reduced biomarker protein or nucleic acid levels. The term “elevated levels” or “higher levels” as used herein refers to levels of a biomarker protein or nucleic acid that are higher than what would normally be observed in a comparable sample from control or normal subjects (i.e., a reference value). In some embodiments, “control levels” (i.e., normal levels) refer to a range of biomarker protein or nucleic acid levels that would normally be expected to be observed in a mammal that does not have a disease. A control level may be used as a reference level for comparative purposes. “Elevated levels” refer to biomarker protein or nucleic acid levels that are above the range of control levels. The ranges accepted as “elevated levels” or “control levels” are dependent on a number of factors. For example, one laboratory may routinely determine the level of biomarker protein or nucleic acid in a sample that are different than the level obtained for the same sample by another laboratory. Also, different assay methods may achieve different value ranges. Value ranges may also differ in various sample types, for example, different body fluids or by different treatments of the sample. One of ordinary skill in the art is capable of considering the relevant factors and establishing appropriate reference ranges for “control values” and “elevated values” of the present invention. For example, a series of samples from control subjects and subjects diagnosed with cancer can be used to establish ranges that are “normal” or “control” levels and ranges that are “elevated” or “higher” than the control range.

Similarly, “reduced levels” or “lower levels” as used herein refer to levels biomarker NEK2 protein or nucleic acid that are lower than what would normally be observed in a comparable sample from control or normal subjects (i.e., a reference value). In some embodiments, “control levels” (i.e. normal levels) refer to a range of biomarker protein or nucleic acid levels that would be normally be expected to be observed in a mammal that does not have a disease and “reduced levels” refer to biomarker protein or nucleic acid levels that are below the range of such control levels.

As used herein, the phrase “difference of the level” refers to differences in the quantity of a particular marker, such as a biomarker protein or nucleic acid, in a sample as compared to a control or reference level. For example, the quantity of particular protein or nucleic acid may be present at an elevated amount or at a decreased amount in samples of patients with a disease compared to a reference level. In one embodiment, a “difference of a level” may be a difference between the level of biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the level of the biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

The biomarker protein or nucleic acid level in a test sample can be used in conjunction with clinical factors other than the biomarker to diagnose a disease. Clinical factors of particular relevance in the diagnosis of cancer include, but are not limited to, the patient's medical history, a physical examination of the patient, complete blood count, cytogenetics, etc.

### Prophylactic and Therapeutic Uses of NEK2 Inhibitors.

In one aspect, the present disclosure provides a method for treating myeloma, lung or breast cancer in a subject, the method comprising administering to the subject an effective amount of a NEK2 inhibitor, e.g., the kinase inhibitor KP372-1. In some embodiments, the myeloma, lung or breast cancer is a drug-resistant cancer. Specifically, the disclosure provides for both prophylactic and therapeutic methods of treating a subject at risk of (or susceptible to) cancer. Accordingly, the present methods provide for the prevention and/or treatment of cancer in a subject by administering an effective amount of the a NEK2 inhibitor to a subject in need thereof. In therapeutic applications, compositions or medicaments are administered to a subject suspected of, or already suffering from such a disease in an amount sufficient to cure, or at least partially arrest, the symptoms of the disease, including its complications and intermediate pathological phenotypes in development of the disease.

In various embodiments, suitable in vitro or in vivo assays are performed to determine the effect of a NEK2 inhibitor and whether its administration is indicated for treatment. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Any method known to those in the art for contacting a cell, organ or tissue with a compound may be employed. In vivo methods typically include the administration of a NEK2 inhibitor, such as those described above, to a mammal, suitably a human. When used in vivo for therapy, the inhibitors are administered to the subject in effective amounts (i.e., amounts that have desired therapeutic effect). The dose and dosage regimen will depend upon the degree of the ophthalmic condition in the subject, the characteristics of the particular compound used, e.g., its therapeutic index, the subject, and the subject's history. The effective amount may be determined during pre-clinical trials and clinical trials by methods familiar to physicians and clinicians. An effective amount of an inhibitor useful in the methods may be administered to a mammal in need thereof by any of a number of well-known methods for administering pharmaceutical compounds. For example, the inhibitor may be administered systemically or locally.

The NEK2 inhibitors described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course.

### EXAMPLES

The present invention is further illustrated by the following examples, which should not be construed as limiting in any way. The following is a description of the materials and methods used throughout the examples.

Study Subjects. Nineteen primary multiple myeloma (MM) patients including 59 samples at baseline, after chemotherapy (pre-1^{st} and pre-2^{nd} bone marrow transplant, and pre-consolidation) were obtained from Huntsman Cancer Institute, University of Utah according to the ARUP protocol 25009.

Gene Expression Profiling). Plasma cell purifications and gene expression profiling, using the Affymetrix U133Plus2.0 microarray, were performed as previously described in Zhan, F., et al. Blood 108, 2020-2028 (2006) and Shaughnessy, J. D., Jr., et al. Blood 109, 2276-2284 (2007). Signal intensities were preprocessed and normalized by GCOS1.1 software (Affymetrix). 59 myeloma cell samples from 19 newly diagnosed myeloma patients in the University of Utah (AUTP-25009 protocol) were used in this study.

Real-time Reverse Transcriptase-PCR (RT-PCR). RNA was extracted using the RNAeasy Kit (Qiagen, Valencia, Calif.). First-strand cDNA was synthesized from 1 μg purified RNA using SuperScript® III (Invitrogen, Carlsbad, Calif.). Primers for NEK2 amplification were obtained from Applied Biosystems (Foster City, Calif.). Real-time PCR was performed on an ABI PRISM 7900 analytical thermal cycler (Applied Biosystems) following the manufacturer's instructions. The glyceraldehyde-3-phosphate dehydrogenase (GAPDH) housekeeping gene was used to normalize samples for RNA quality and quantity. The ΔΔCt approach was used for relative quantification of NEK2 mRNA. Each sample was analyzed in triplicate and the results were expressed as the mean±SEM.

Western Blots. Western blots were performed to examine the protein levels in MM cells as previously described in Xiong, W., et al. Blood 112, 4235-4246 (2008) and Zhan, F., et al. Blood 109, 4995-5001 (2007). NEK2 antibodies were purchased from BD Biosciences Pharmingen (San Jose, Calif.); and all other primary antibodies were purchased from Cell Signaling Technology (Beverly, Mass.). β-actin was used to normalize the amount of protein in each sample.

Cell proliferation assay. Briefly, cancer cells transfected with NEK2 cDNA or NEK2 shRNA and their relative controls of empty vector or Scrambled shRNA were seeded in 12-well plates at 0.2×10⁶ per well. All tests were set in triplicate or quadruplicate. Cultured cells were counted with a hemocytometer to evaluate cellular proliferation, and dead cells were determined by trypan blue staining, from which dead-cell fraction was calculated. Cell proliferation assays were done at various time points for at least one week after infection or addition doxycycline one day.

Treatment of NEK2-overexpressing MM cells with chemotherapeutic drugs. To determine the role of NEK2 in cancer drug-resistance, ARK, KMS28PE, OCI-MY5, H1299, and MCF7 cell lines were transfected with NEK2-cDNA to overexpress NEK2 by lentivirus expression vector system. 1×10⁶ cells were treated with bortezomib at 5 nM for 48 hours in the cultures. Cell proliferation was evaluated by cell counting with a hemocytometer, and dead cells were determined by trypan blue staining, from which dead-cell fraction was calculated. Untreated cells and empty-vector (EV)-transfected cells with or without drug-treatment were used as controls. Similar experiments were performed for doxorubicin (100 nM) and etoposide (100 nM), and cells were treated for 48 hours.

Statistical analysis. Student t test (=2 groups) and one-way ANOVA (>3 groups) were used to compare various experimental groups. The Kaplan-Meier method was used to estimate event-free survival (EFS), overall survival (OS), and post-relapse survival. Significance was set at P<0.05.

NEK2 kinase activity assay. Purified recombinant NEK2 kinase protein (Cell Signaling Technology, Beverley, Mass.), was added to 96 well plates in the appropriate reaction buffer. Compounds were added to each well to a final concentration of 5 μM. Reaction mixtures were incubated for 15 minutes. The Kinase-Glo Luminescent Kinase Assay kit (Promega, Madison, Wis.) was used to determine the remaining ATP, which is inversely related to kinase activity.

Efficacy of KP372-1 in a mouse model of multiple myeloma. C57BL/KaLwRij mice (n=5 in each group) were injected with 1×10⁶ 5TGM1 cells through their tail vein. Seven days following tumor inoculation, tumor-bearing mice were treated with KP372-1, bortezomib or a combination of KP372-1 with bortezomib.

### Example 1

### NEK2 is a Biomarker for Drug Resistance and Relapse in Multiple Myeloma and Other Cancers

We first compared gene expression profiles from 19 paired myeloma cell samples collected at diagnosis and again after induction chemotherapy (before the first stem cell transplant). A total of 615 probe sets were significantly differentially expressed (P<0.05 and average fold change>1.5). Similarly, we compared the gene expression profiles of 51 paired myeloma samples collected at baseline and at early relapse; 864 genes were differentially expressed (P<0.05 and average fold change>1.5). By intersection analyses of these 2 comparisons, we identified 56 genes, the expressions of which were significantly up-regulated both after chemotherapy and at relapse (Table 1). The 56 genes include kinases (TK1, CDKN3, PTPRJ, PTPRC, TYROBP, SYK, and NEK2), DNA synthesis and chromosome assembly genes (CDC2, TOP2A, TYMS, CCNA2, CCNB1, PBK, AURKA, KIF4A, NUSAP1, ARRB2, and KNTC2), oncogene and programmed cell death/apoptotic genes (BIRC5, RHOC, OCC-1, and DEK), B cell differentiation and immunology-related genes (HLA-DRA, HLA-DRB1, IFI30, FCGR3B, and FCER1G), genes associated with cell adhesion and metastasis (ITGB2, HBEGF, LMNB1, S100A8, S100A9, MMP9, and FGL2), polycomb-like genes (EZH2 and PHF19), and genes related to metabolism and renal amyloidosis (G6PD and LYZ). These results suggest that these 56 genes may be functionally related to drug-resistance in MM.

TABLE 1
56 genes over-expressed after chemotherapy and at relapse in MM

| | | | Pre-1stBMT | | | | Overall | |
|---|---|---|---|---|---|---|---|---|
| | | | vs. | | Relapse vs. | | Survival | |
| | Gene | Chromosomal | Baseline | | Baseline | | in TT2 | |
| Probe Set | Symbol | Location | P value | Ratio | P value | Ratio | P value | HR |
| 204641_at | NEK2 | 1q32.2-q41 | 0.0166 | 1.68 | 0.00028 | 1.69 | 2.06 × 10⁻¹³ | 5.01 |
| 225834_at | FAM72A | 1p12 | 0.0001 | 1.59 | 1.26 × 10⁻⁵ | 1.58 | 9.9 × 10⁻¹¹ | 4.97 |
| 202095_s_at | BIRC5 | 17q25 | 0.0085 | 1.71 | 2.14 × 10⁻⁷ | 2.19 | 8.12 × 10⁻⁹ | 4.60 |
| 204033_at | TRIP13 | 5p15.33 | 0.0097 | 1.57 | 0.00015 | 1.50 | 1.53 × 10⁻⁸ | 4.52 |
| 214710_s_at | CCNB1 | 5q12 | 0.0053 | 1.53 | 0.00021 | 1.53 | 2.4 × 10⁻⁹ | 4.46 |
| 208079_s_at | AURKA | 20q13.2-q13.3 | 0.0011 | 1.66 | 1.28 × 10⁻⁵ | 1.57 | 1.52 × 10⁻⁸ | 4.45 |
| 227212_s_at | PHF19 | 9q33.2 | 0.0051 | 1.81 | 5.45 × 10⁻⁷ | 1.99 | 8.84 × 10⁻⁸ | 4.32 |
| 228273_at | — | — | 0.0004 | 1.69 | 5.41 × 10⁻⁵ | 1.55 | 9.99 × 10⁻⁷ | 3.87 |
| 218542_at | CEP55 | 10q23.33 | 0.0032 | 1.58 | 8.73 × 10⁻⁵ | 1.60 | 1.52 × 10⁻⁹ | 3.56 |
| 223381_at | NUF2 | 1q23.3 | 0.0055 | 1.69 | 0.00673 | 1.58 | 7.31 × 10⁻⁹ | 3.54 |
| 201291_s_at | TOP2A | 17q21-q22 | 0.0198 | 1.55 | 1.61 × 10⁻⁵ | 1.71 | 2.83 × 10⁻⁷ | 3.41 |
| 218663_at | NCAPG | 4p15.33 | 0.0301 | 1.62 | 9.81 × 10⁻⁵ | 1.69 | 5.38 × 10⁻⁶ | 2.89 |
| 202870_s_at | CDC20 | 1p34.1 | 0.0053 | 1.68 | 3.82 × 10⁻¹⁸ | 1.95 | 7.12 × 10⁻⁶ | 2.86 |
| 202589_at | TYMS | 18p11.32 | 0.0013 | 1.64 | 6.82 × 10⁻⁷ | 1.83 | 5.35 × 10⁻⁶ | 2.79 |
| 219978_s_at | NUSAP1 | 15q15.1 | 0.0066 | 1.55 | 4.60 × 10⁻⁷ | 1.87 | 2.91 × 10⁻⁵ | 2.58 |
| 1554408_a_at | TK1 | 17q23.2-q25.3 | 0.0358 | 1.64 | 2.78 × 10⁻⁵ | 1.71 | 9.48 × 10⁻⁵ | 2.50 |
| 219148_at | PBK | 8p21.2 | 0.0280 | 1.57 | 0.000129 | 1.62 | 0.00019 | 2.45 |
| 218355_at | KIF4A | 5q33.1 | 0.0018 | 1.85 | 0.001267 | 1.54 | 1.49 × 10⁻⁵ | 2.30 |
| 209773_s_at | RRM2 | 2p25-p24 | 0.0048 | 1.87 | 9.23 × 10⁻⁶ | 1.60 | 0.00064 | 2.11 |
| 205099_s_at | CCR1 | 3p21 | 0.0493 | 1.77 | 0.000129 | 1.63 | 0.0255 | 2.07 |
| 242907_at | — | — | 0.0013 | 1.77 | 0.020856 | 0.60 | 0.014 | 2.04 |
| 203418_at | CCNA2 | 4q25-q31 | 0.0025 | 1.55 | 0.001845 | 1.57 | 0.0023 | 2.00 |
| 217388_s_at | KYNU | 2q22.2 | 0.0005 | 4.26 | 1.47 × 10⁻⁵ | 3.03 | 0.046 | 1.87 |
| 212192_at | KCTD12 | 13q22.3 | 0.0268 | 2.07 | 0.007109 | 1.60 | 0.0048 | 1.74 |
| 203276_at | LMNB1 | 5q23.3-q31.1 | 0.0027 | 1.55 | 6.74 × 10⁻⁶ | 1.75 | 0.016 | 1.69 |
| 203821_at | HBEGF | 5q23 | 0.0361 | 1.95 | 0.00113 | 2.27 | 0.029 | 1.65 |
| 225105_at | OCC-1 | 12q23.3 | 0.0154 | 1.82 | 0.001757 | 1.58 | 0.0144 | 1.58 |
| 208891_at | DUSP6 | 12q22-q23 | 0.0074 | 2.35 | 0.010375 | 1.54 | 0.19 | 0.77 |
| 204466_s_at | SNCA | 4q21 | 0.0094 | 1.60 | 0.0297304 | 2.18 | 0.13 | 0.75 |
| 202275_at | G6PD | Xq28 | 0.0074 | 2.05 | 0.001487 | 1.75 | 0.2 | 0.74 |
| 207857_at | LILRA2 | 19q13.4 | 0.0004 | 5.15 | 0.002561 | 2.12 | 0.2 | 0.68 |
| 208636_at | ACTN1 | 14q24.1-q24.2 | 0.0371 | 2.67 | 0.004157 | 1.71 | 0.092 | 0.68 |
| 224964_s_at | GNG2 | 14q21 | 0.0026 | 1.69 | 0.015271 | 1.56 | 0.044 | 0.68 |
| 212588_at | PTPRC | 1q31-q32 | 0.0069 | 2.41 | 0.001521 | 1.68 | 0.054 | 0.67 |
| 203936_s_at | MMP9 | 20q11.2-q13.1 | 0.0032 | 6.35 | 0.001704 | 1.51 | 0.02 | 0.58 |
| 203388_at | ARRB2 | 17p13 | 0.0391 | 1.58 | 6.98 × 10⁻⁵ | 1.55 | 0.0031 | 0.56 |
| 203535_at | S100A9 | 1q21 | 0.0457 | 5.15 | 1.78 × 10⁻⁵ | 3.27 | 0.00197 | 0.56 |
| 226757_at | IFIT2 | 10q23-q25 | 0.0065 | 1.71 | 0.002407 | 1.88 | 0.0023 | 0.56 |
| 204834_at | FGL2 | 7q11.23 | 0.0132 | 2.13 | 3.29 × 10⁻⁵ | 1.87 | 0.0017 | 0.55 |
| 204007_at | FCGR3B | 1q23 | 0.0052 | 4.82 | 0.0011267 | 2.00 | 0.0016 | 0.53 |
| 231688_at | — | — | 0.0079 | 4.27 | 0.012985 | 2.21 | 0.016 | 0.53 |
| 203973_s_at | CEBPD | 8p11.2-p11.1 | 0.0072 | 5.13 | 7.67 × 10⁻⁷ | 2.47 | 0.0011 | 0.52 |
| 201422_at | IFI30 | 19p13.1 | 0.0012 | 1.92 | 0.000102 | 1.87 | 0.061 | 0.52 |
| 202803_s_at | ITGB2 | 21q22.3 | 0.0128 | 4.98 | 2.59 × 10⁻¹⁴ | 2.33 | 0.00088 | 0.52 |
| 213975_s_at | LYZ | 12q15 | 0.0357 | 2.70 | 5.60 × 10⁻⁵ | 2.65 | 7.79 × 10⁻⁵ | 0.48 |
| 205863_at | S100A12 | 1q21 | 0.0054 | 5.42 | 3.66 × 10⁻⁶ | 3.34 | 0.0048 | 0.46 |
| 202917_s_at | S100A8 | 1q21 | 0.0433 | 2.70 | 2.67 × 10⁻⁵ | 2.57 | 0.00032 | 0.45 |
| 204232_at | FCER1G | 1q23 | 0.0034 | 3.58 | 0.00017 | 1.79 | 5.4 × 10⁻⁵ | 0.44 |
| 208306_x_at | HLA- | 6p21.3 | 0.0044 | 1.72 | 1.09 × 10⁻⁸ | 3.15 | 0.00013 | 0.44 |
| | DRB1 | | | | | | | |
| 208894_at | HLA- | 6p21.3 | 0.0135 | 1.54 | 0.000219 | 1.94 | 0.0028 | 0.41 |
| | DRA | | | | | | | |
| 210629_x_at | LST1 | 6p21.3 | 0.0051 | 2.49 | 0.005881 | 2.07 | 0.00011 | 0.38 |
| 211990_at | HLA- | 6p21.3 | 0.0229 | 1.61 | 5.06 × 10⁻⁶ | 2.02 | 0.00016 | 0.37 |
| | DPA1 | | | | | | | |
| 212543_at | AIM1 | 6q21 | 0.0038 | 2.50 | 0.004038 | 1.61 | 0.00052 | 0.36 |
| 227266_s_at | FYB | 5p13.1 | 0.0038 | 3.25 | 0.004901 | 1.65 | 1.25 × 10⁻⁵ | 0.34 |
| 228532_at | C1orf162 | 1p13.2 | 0.0195 | 3.15 | 0.012385 | 2.28 | 0.002 | 0.32 |
| 204122_at | TYROBP | 19q13.1 | 0.0068 | 4.61 | 4.03 × 10⁻⁶ | 2.21 | 4.11 × 10⁻⁶ | 0.28 |

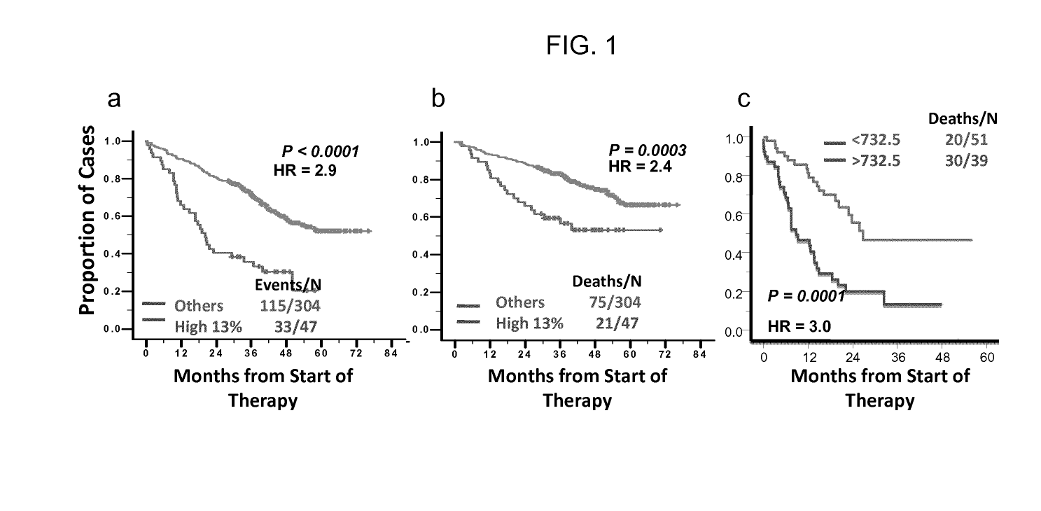

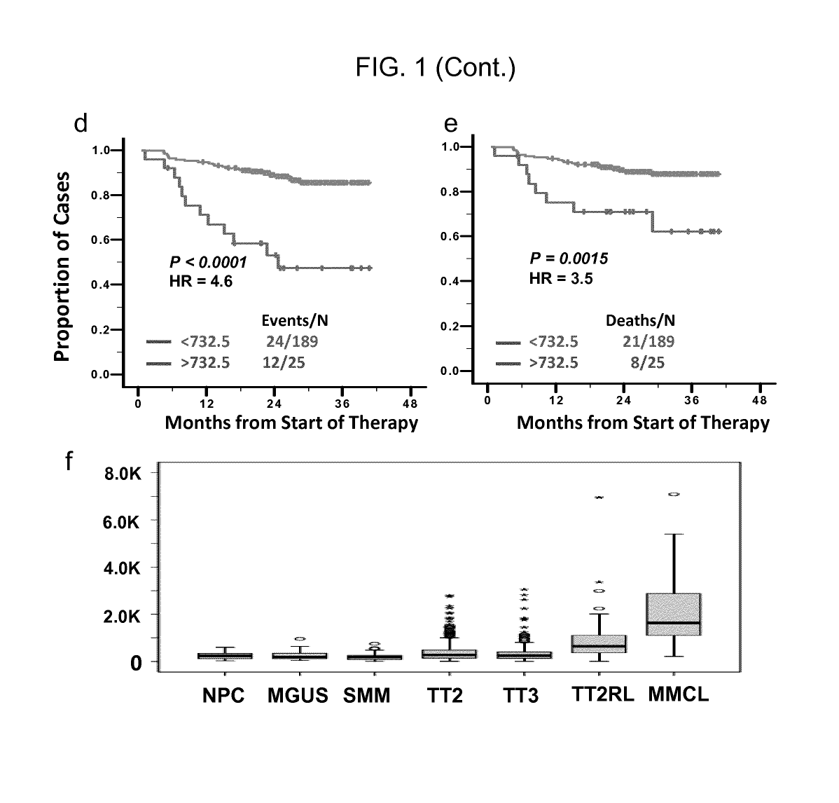

NEK2 is the most significant gene linked to an aggressive phenotype in MM and other cancers. To examine the functional role of these 56 genes in MM progression, we correlated the gene expression of these 56 genes with clinical outcomes. A Kaplan-Meier survival analysis was performed on the Total Therapy 2 (TT2) cohort, which included 351 newly diagnosed myelomas. The correlation between gene expression and survival was determined by the P value and hazard ratio (HR) at the best expression signal cut-off after a permutation test. NEK2 was the gene most strongly associated with inferior survival in unadjusted log rank tests. We recently reported a 70 high-risk gene model, in which a high-risk score was present in 13% of myeloma patients with short durations of event-free survival (EFS) and overall survival (OS). By using the same proportion cut-off of patients (13%), high NEK2 expression was associated with inferior EFS and OS (FIG. 1a, b, P<0.0001 and P=0.0003, respectively) and post-relapse survival of TT2 patients (FIG. 1c, P=0.0001), as well as in TT3 (FIG. 1d, e, P<0.0001 and P=0.0015, respectively). On both univariate and multivariate analyses of clinical characteristics affecting OS, NEK2 gene expression represented an independent factor associated with poor prognosis in TT2 (Table 2).

TABLE 2
Univariate and multivariate analyses of clinical characteristics
affecting OS by NEK2 expression in TT2

| | | Low | |
|---|---|---|---|
| | | NEK2 | |
| | High NEK2 | (%, | |
| Characteristics | (%, n = 47) | n = 304) | P-value^{†} |
| Age at least 65 y | 19.1 | 22.4 | NS |
| Female sex | 38.3 | 44.1 | NS |
| White race | 95.7 | 87.5 | NS |
| IgA isotype | 31.9 | 25.0 | NS |
| CRP at least 4.0 mg/L | 11.1 | 5.3 | NS |
| β2-Microglobulin at least 4.0 mg/L | 44.7 | 32.9 | NS |
| Hemoglobin less than 10 g/dL | 34.1 | 24.0 | NS |
| Bone marrow plasma cells | 47.4 | 53.4 | NS |
| (by aspiration) 33% or greater | | | |
| Albumin less than 3.5 g/dl | 51.1 | 34.2 | 0.020 |
| Creatinine at least 2.0 mg/dL | 22.7 | 9.8 | 0.017 |
| (221 μmol/L) | | | |
| MRI focal bone lesions, at least 3 | 76.6 | 55.7 | 0.005 |
| LDH at least 190 IU/L | 53.3 | 31.3 | 0.004 |
| Chromosomal abnormalities | 66.0 | 30.6 | <0.001 |
| (by G-banding) | | | |
| hyperdiploid | 23.4 | 17.8 | NS |
| hypodiploid | 42.6 | 11.2 | <0.001 |
| Deletion of chromosome 13 | 72.1 | 46.1 | 0.001 |
| Amplification of 1q21 | 85.4 | 37.3 | <0.001 |
| High-risk model (17-gene)* | 59.6 | 5.9 | <0.001 |
| Subgroups with poor prognosis | 88.6 | 25.9 | <0.001 |
| (PR/MS/MF)* | | | |
| Proliferation (PR)* | 42.6 | 3.0 | <0.001 |
| Multivariate analysis of | | | |
| clinical characteristics affecting OS | | | |
| NEK2 high 13% | | | 0.022 |
| Amplification of 1q21 | | | 0.015 |
| Creatinine at least 2.0 mg/dL | | | 0.007 |
| High-risk model (17-gene)* | | | 0.002 |

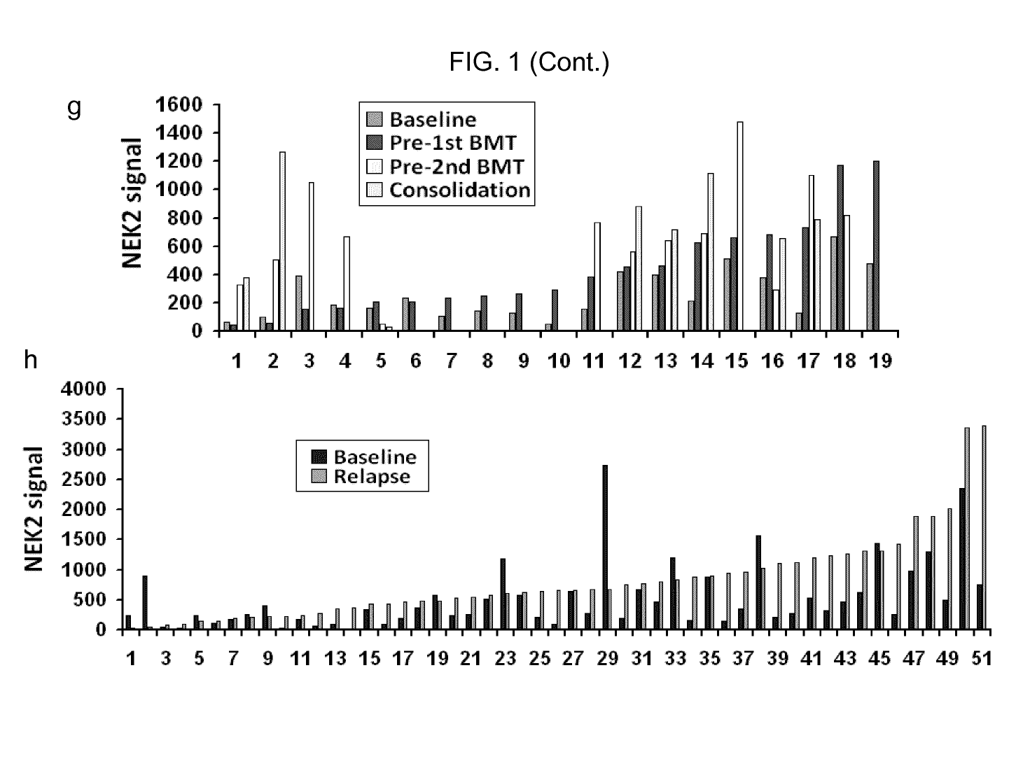

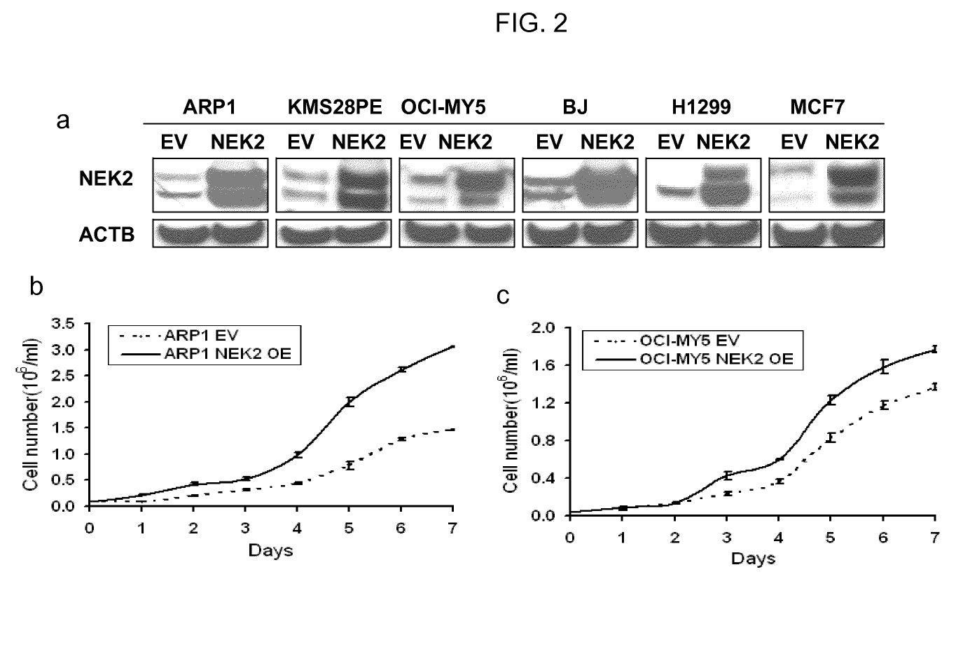

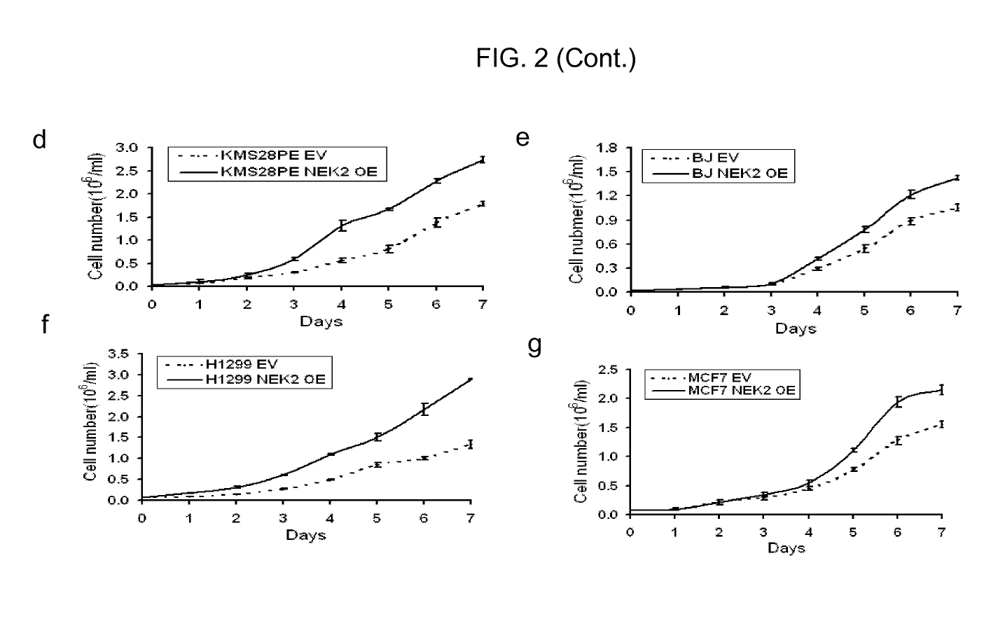

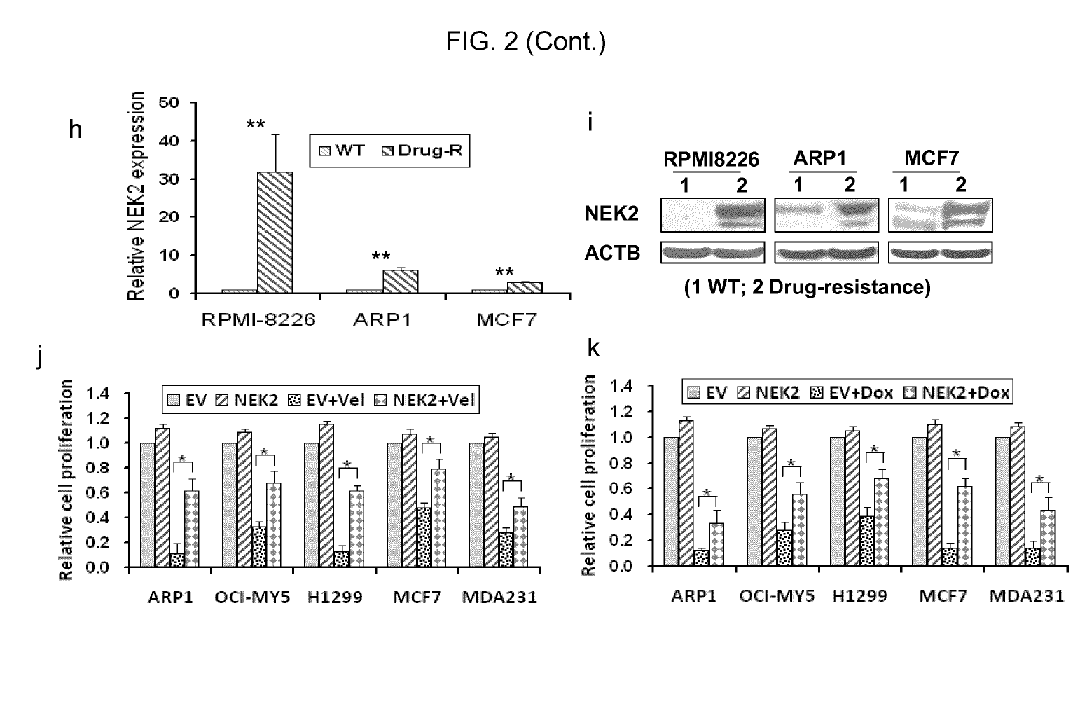

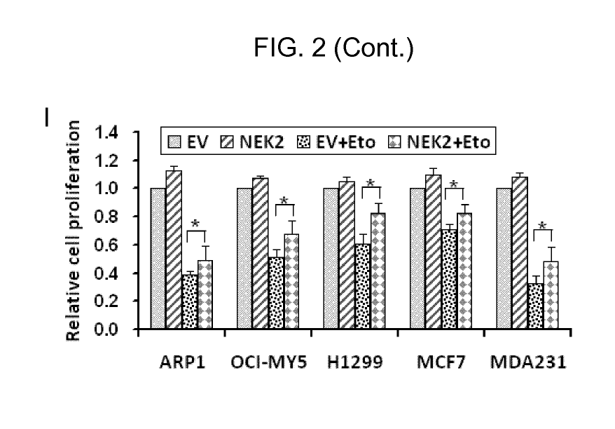

We then compared the NEK2 expression in plasma cell samples from healthy donors (NPC), patients with MGUS, patients with smoldering myeloma (SMM), patients with newly diagnosed myeloma entered on TT2 and TT3, relapse patients on TT2 (TT2 RL) and myeloma cell lines (MMCL). As shown in (FIG. 1f), NEK2-expression was very low or undetectable in NPC, and progressively increased in MGUS, SMM, MM, TT2 RL and MMCL. Gene expression profiling data of myeloma plasma cells from other datasets GSE6205, GSE2113 and GSE6477 showed similar results with low or undetectable expression of NEK2 in plasma cell samples from healthy donors with progressively increased expression in cell samples from MGUS, SMM, MM and MMCL (data not shown). Among the 19 paired myeloma samples at baseline and after chemotherapy (pre-first stem cell transplant), NEK2 expression increased at least 20% in 12 of the 19 samples (FIG. 1g; P=0.0166). Importantly, the serial gene expression profiles obtained pre-second transplant, and pre-consolidation, showed either continuing increase of NEK2 expression or stable high levels of NEK2 (FIG. 2g). By analyzing the 51 paired gene expression profiles at baseline and relapse, 40 of 51 patients had increased NEK2 expression at relapse (FIG. 1h; P=0.007). These results indicate that NEK2 expression is associated with an aggressive phenotype in MM.

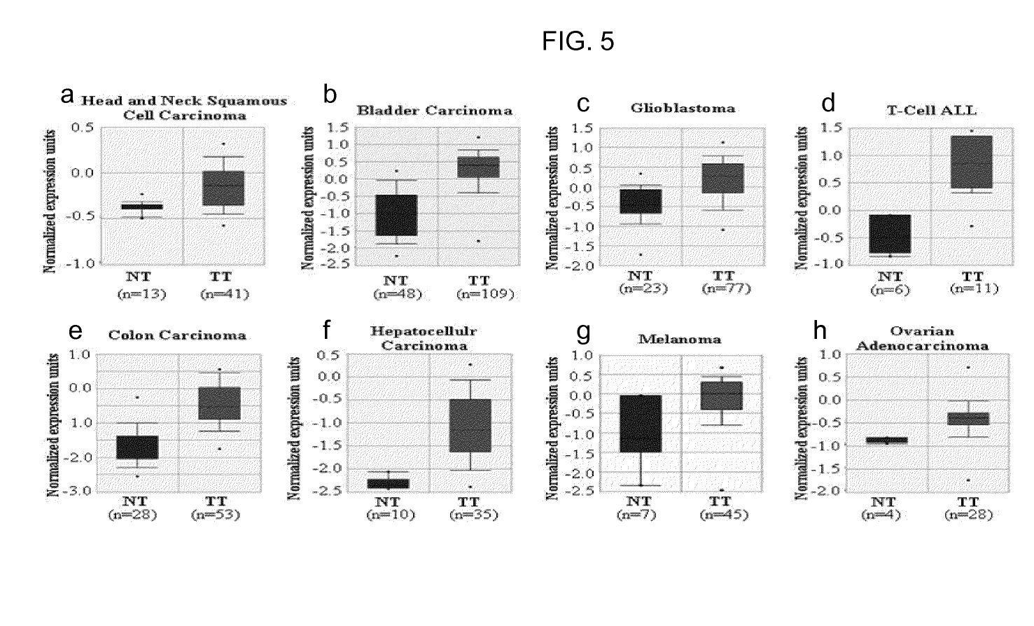

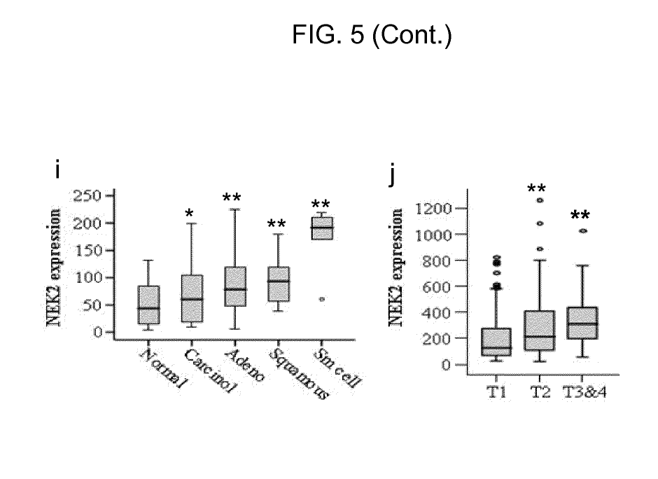

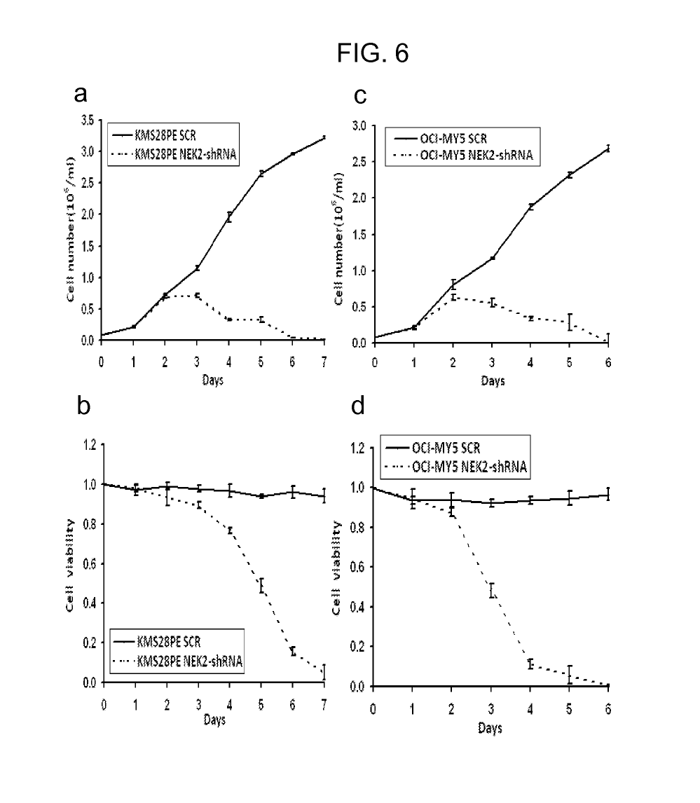

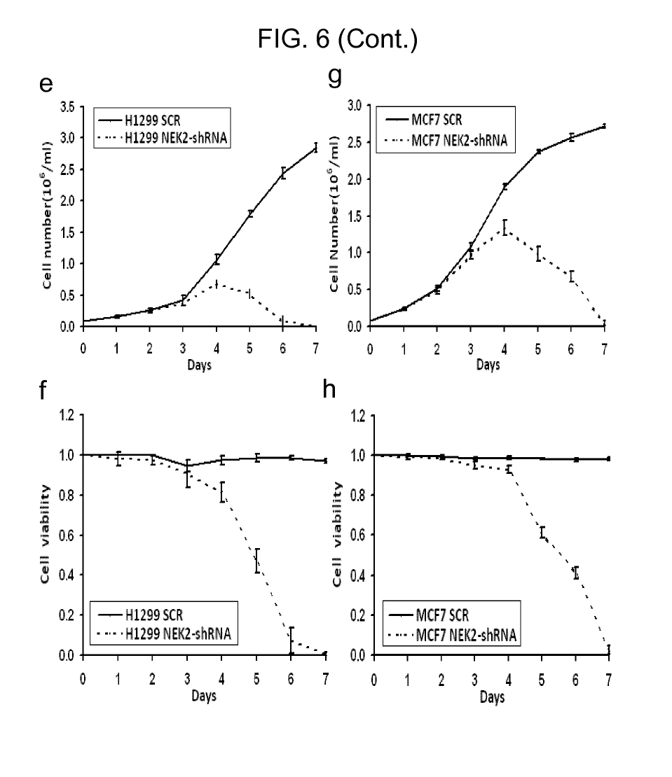

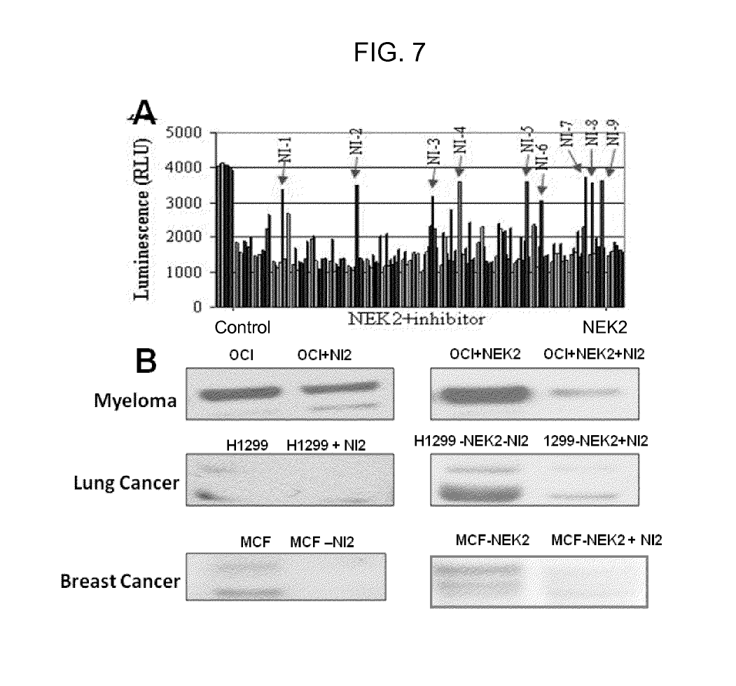

We subsequently examined the clinical implication of high NEK2 expression in other cancer types. We used microarray datasets and the associated clinical information of seven different cancers, including acute myeloid leukemia, bladder cancer, breast cancer, glioma, lung adenocarcinoma, mantle cell lymphoma, and mesothelioma. We found that over-expression of NEK2 conferred an inferior survival in all of the seven cancers (FIGS. 6a-g; P<0.0001). These results demonstrated that NEK2 over-expression is related to poor prognosis in multiple cancers. We also investigated NEK2 gene expression in other human cancers. NEK2 expression was significantly up-regulated compared to the normal counterpart in all of the examined cancers, including head and neck squamous cell carcinoma (FIG. 5a), bladder carcinoma (FIG. 7b), glioblastoma (FIG. 7c), T-cell acute lymphoblastic leukemia (FIG. 5d), colon carcinoma (FIG. 5e), hepatocellular carcinoma (FIG. 5f), melanoma (FIG. 5g), ovarian adenocarcinoma (FIG. 5h), and lung cancers. The lung gene expression profile dataset included specimens of normal lung (n=17), pulmonary carcinoids (n=20), lung adenocarcinomas (n=127), squamous cell lung carcinomas (n=21), and small cell lung cancer specimens (SCLC) (n=6). FIG. 5i shows the median expression levels of NEK2 were 55, 68, 85, 96, and 174, respectively. SCLC is the most aggressive tumor type in lung cancer and has the highest NEK2 expression (P=0.005). The correlation analysis of NEK2 gene expression and TNM staging in lung adenocarcinoma showed that the median NEK2 expression level in stage I was 204 (range 26-826), while in stage 1I and III-IV it was 269 (range 25-1264) and 337 (range 57-1029), respectively (FIG. 5j; P<0.05).

Increased NEK2 expression promotes cell proliferation and drug resistance in cancer. To test the role of NEK2 in promoting cell growth, we over-expressed NEK2 in normal fibroblasts BJ, the MM cell lines ARP1, KMS28PE, and OCI-MY5, the lung cancer cell line H1299, and breast cancer cell line MDA231 by lentivirus-mediated NEK2-cDNA transfection (FIG. 2a). NEK2 over-expression significantly increased cell proliferation in both normal and cancer cells compared to empty vector (EV)-transfected cells (FIGS. 2b-2g). We subsequently examined the role of NEK2 in myeloma drug-resistance. We developed 2 bortezomib-resistant MM cell lines, RPMI 8226 and ARP1, by a stepwise increase of the bortezomib concentration in cultures. The IC₅₀s of bortezomib treatment in bortezomib-resistant RPMI8226 and ARP1 cells were 250.12 nM and 25.76 nM, respectively, whereas the IC₅₀s of the parental cells were 4.75 nM and 7.47 nM, respectively (data not shown).

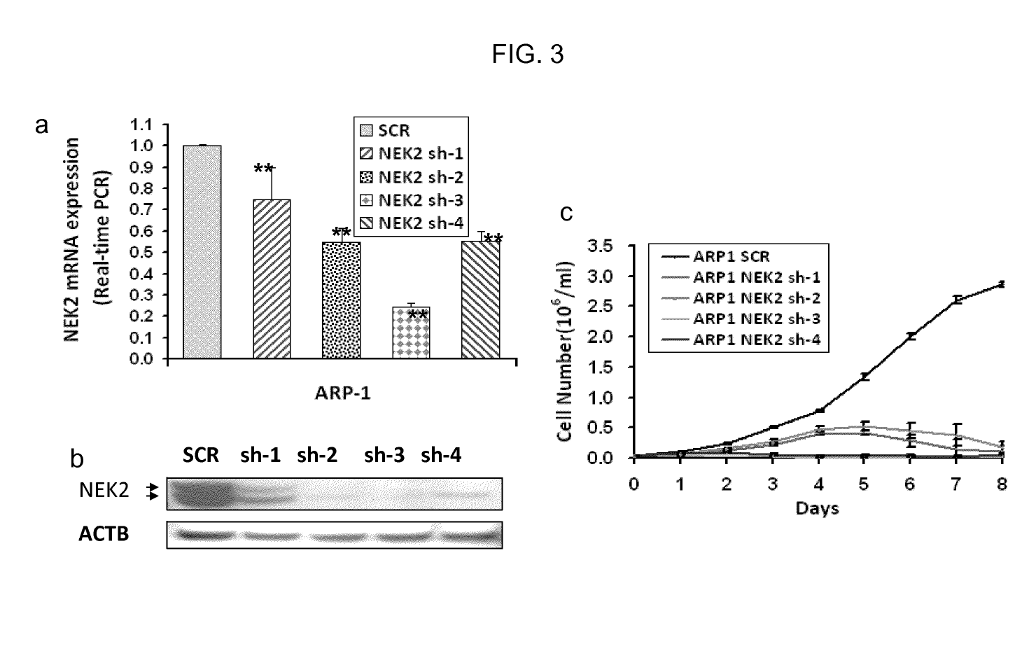

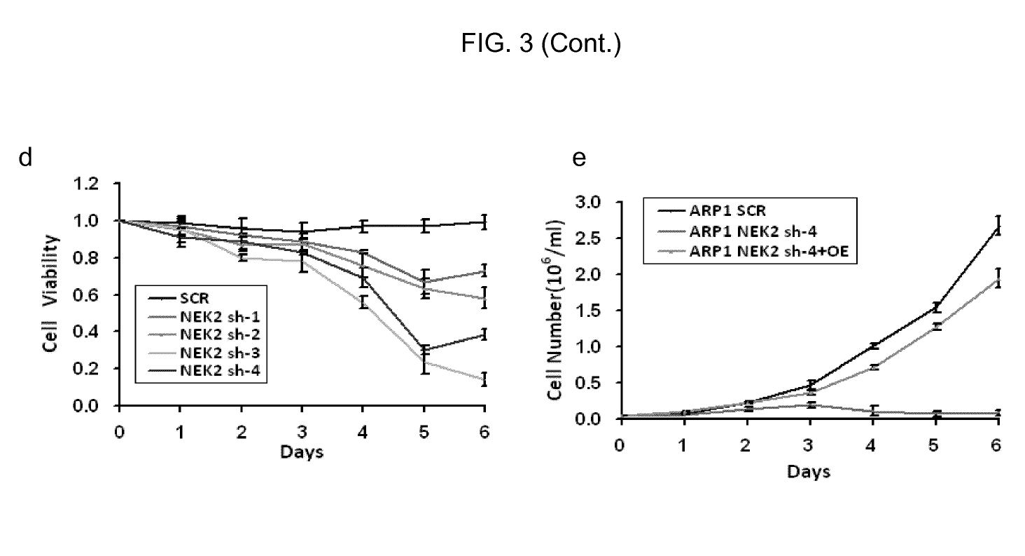

Real time-PCR detected significantly higher levels of NEK2 mRNA in drug-resistant RPMI8226, ARP1 and MCF7/MDR cells than in the wild-type counterparts (FIG. 2h). Western-blots further confirmed the increased NEK2 proteins in the drug-resistant cells (FIG. 2i). To determine whether NEK2 over-expression is sufficient to confer drug-resistance in cancer cells, NEK2-transfected ARP1, KMS28PE, H1299, MCF7, and MDA231 cells were then treated with bortezomib at the dose of 4, 8, and 16 nM bortezomib for 48 hours and cell death was evaluated. EV-transfected cells with or without bortezomib served as controls. As shown in FIG. 3j, 8 nM bortezomib treatment induced significantly less growth inhibition in NEK2-transfected cells compared with EV-transfected controls (P<0.05). Similarly, treatment of doxorubicin (100 nM) (FIG. 2k) and etoposide (100 nM) (FIG. 21) for 48 hours, induced significantly less growth inhibition and cell death (data not shown) in NEK2-transfected cells compared with EV-transfected controls (P<0.05).

### Example 2

### Inhibition of NEK2 Induces Apoptosis in Tumor Cells

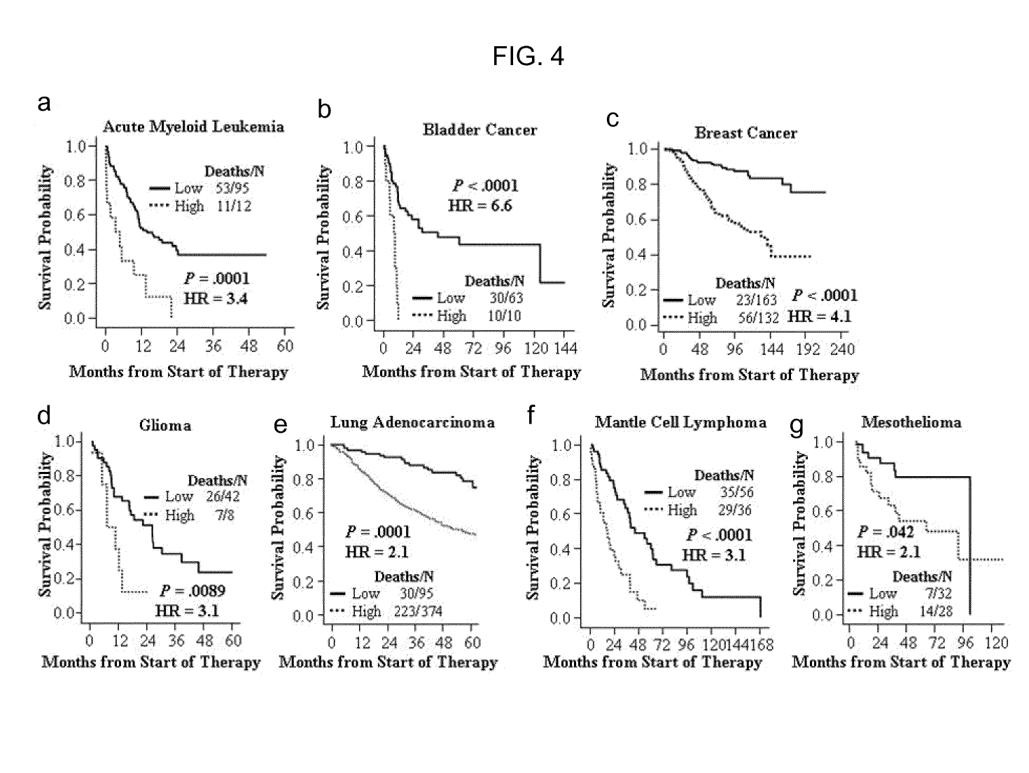

To examine the function of NEK2 in maintaining cancer cell survival, specific sh-RNAs (using lentivirus shRNA expression vector system) were used to knockdown NEK2 in tumor cells. We designed 4 shRNAs against NEK2 gene (sh1-4); sh1-3 target the NEK2 coding region and sh4 targets the 3′ untranslated region (UTR). Real-time PCR and Western-blots confirmed the remarkable down-regulation of NEK2 proteins in ARP1 cells after transfection of these NEK2-shRNAs, especially the sh3 (FIGS. 3a, b). NEK2-shRNA-induced growth inhibition and cell death were examined daily. Cells were cultured for 8 days and non-target scramble-transfected cells were used as controls. As shown in FIGS. 3c, d, all four NEK2-shRNAs induced significantly growth inhibition (FIG. 3c) and cell death (FIG. 3d) compared to the scramble controls; sh3 and sh4 appeared more potent (FIGS. 3c, d) than sh1 and sh2. To address the issue of off-target toxicities, we doubly-transfected ARP1 cells with NEK2-sh4 and the NEK2-cDNA, which contained only the coding region of NEK2, and cell growth was examined. SCR-transfected cells and NEK2-sh4-transfected cells were used as controls. As shown in FIG. 4e, forced over-expression of NEK2 successfully abrogated cell growth inhibition induced by sh4 in ARP1 cells, demonstrating that the cytotoxic effects induced by sh4 were directly related to specific knockdown of NEK2. We then chose our most potent inhibitor NEK2-sh3 to knockdown NEK2 in other cancer cells, including KMS28PE and OCI-MY5 myeloma cells, NCI-H1299 lung cancer cells and MCF7 breast cancer cells. Western blots confirmed the efficient knockdown of NEK2 in these cells (not shown). FIG. 6 shows that NEK2-knockdown induced significant cell growth inhibition at day 4 in KMS28PE, OCI-MY5, H1299 and MCF7 cells compared to the scrambled controls.

It is noteworthy that the 51 genes related to either over-expression or silenced NEK2 have a defined function in DNA replication, cell cycle progression, chromosome condensation, mitosis, and cytokinesis (CDC2, PBK, CENPF, KIF11, TMSL8, RAD51AP1, ESPL1, ECT2, MYBL1, MPHOSPH1, DNA2, GAS2L3, SMC4, TMPO, E2F7, HPSE, MKI67, and OCC-1) (Table 3).

TABLE 3
Genes highly correlated with NEK2 expression

| | | | ARP1 | | | |
|---|---|---|---|---|---|---|
| | | Chromosomal | NEK2- | NEK2- | Primary MM | |
| Probe Set | Gene Symbol | Location | OV | shRNA | P value | High/Low |
| 204641_at | NEK2 | 1q32.2-q41 | 2.11 | 0.07 | 4.38 × 10⁻⁵⁵ | 21.70 |
| 209301_at | CA2 | 8q22 | 2.33 | 0.21 | 0.001169 | 2.48 |
| 222881_at | HPSE | 4q21.3 | 2.09 | 0.22 | 0.001575 | 1.68 |
| 209829_at | C6orf32 | 6p22.3-p21.32 | 2.22 | 0.23 | 0.008407 | 1.68 |
| 204817_at | ESPL1 | 12q | 1.71 | 0.26 | 8.91 × 10⁻¹⁷ | 3.46 |
| 219148_at | PBK | 8p21.2 | 1.65 | 0.29 | 2.02 × 10⁻³³ | 6.31 |
| 205085_at | ORC1L | 1p32 | 1.66 | 0.29 | 6.57 × 10⁻⁹ | 3.35 |
| 201362_at | IVNS1ABP | 1q25.1-q31.1 | 2.02 | 0.31 | 0.002112 | 1.36 |
| 209754_s_at | TMPO | 12q22 | 1.72 | 0.34 | 5.73 × 10⁻⁸ | 2.13 |
| 213906_at | MYBL1 | 8q22 | 1.70 | 0.36 | 1.85 × 10⁻⁵ | 2.63 |
| 204146_at | RAD51AP1 | 12p13.2-p13.1 | 1.56 | 0.38 | 6.7 × 10⁻²⁰ | 4.49 |
| 205235_s_at | MPHOSPH1 | 10q23.31 | 1.71 | 0.38 | 8.29 × 10⁻¹² | 2.35 |
| 203474_at | IQGAP2 | 5q13.3 | 2.74 | 0.38 | 0.00966 | 1.39 |
| 204444_at | KIF11 | 10q24.1 | 1.71 | 0.39 | 1.63 × 10⁻²⁹ | 5.64 |
| 219787_s_at | ECT2 | 3q26.1-q26.2 | 1.61 | 0.40 | 1.55 × 10⁻²² | 2.90 |
| 213647_at | DNA2 | 10q21.3-q22.1 | 1.95 | 0.41 | 4.90 × 10⁻¹⁵ | 2.34 |
| 200783_s_at | STMN1 | 1p36.1-p35 | 1.72 | 0.41 | 1.96 × 10⁻¹⁶ | 1.96 |
| 238529_at | — | — | 1.76 | 0.42 | 2.71 × 10⁻⁶ | 2.56 |
| 205347_s_at | TMSL8 | Xq21.33-q22.3 | 2.66 | 0.43 | 1.20 × 10⁻⁹ | 5.26 |
| 207828_s_at | CENPF | 1q32-q41 | 1.81 | 0.43 | 1.13 × 10⁻²⁴ | 6.03 |
| 225105_at | OCC-1 | 12q23.3 | 2.21 | 0.49 | 0.000105 | 1.52 |
| 224734_at | HMGB1 | 13q12 | 2.18 | 0.49 | 0.001923 | 1.39 |
| 204054_at | PTEN | 10q23.3 | 1.95 | 0.49 | 0.008585 | 1.19 |
| 210543_s_at | PRKDC | 8q11 | 1.78 | 0.50 | 4.05 × 10⁻⁵ | 1.38 |
| 228033_at | E2F7 | 12q21.2 | 1.65 | 0.51 | 1.27 × 10⁻⁹ | 2.09 |
| 203213_at | CDC2 | 10q21.1 | 1.97 | 0.53 | 4.42 × 10⁻³⁰ | 9.23 |
| 227801_at | TRIM59 | 3q26.1 | 2.07 | 0.53 | 0.000193 | 1.39 |
| 221297_at | GPRC5D | 12p13.3 | 2.04 | 0.55 | 0.002706 | 1.44 |
| 212020_s_at | MKI67 | 10q25-qter | 2.01 | 0.56 | 8.55 × 10⁻¹⁰ | 1.63 |
| 204001_at | SNAPC3 | 9p22.3 | 1.85 | 0.57 | 0.000106 | 1.35 |
| 238756_at | GAS2L3 | 12q23.1 | 4.75 | 0.60 | 1.25 × 10⁻¹¹ | 2.33 |
| 209804_at | DCLRE1A | 10q25.1 | 1.59 | 0.61 | 0.010136 | 1.32 |
| 222039_at | LOC146909 | 17q21.31 | 1.70 | 0.61 | 1.69 × 10⁻²² | 2.73 |
| 201663_s_at | SMC4 | 3q26.1 | 1.96 | 0.63 | 2.65 × 10⁻¹³ | 2.21 |
| 201163_s_at | IGFBP7 | 4q12 | 2.25 | 0.63 | 0.005211 | 5.05 |
| 229610_at | CKAP2L | 2q13 | 1.88 | 0.63 | 1.29 × 10⁻¹⁸ | 3.50 |
| 209896_s_at | PTPN11 | 12q24 | 1.60 | 0.63 | 0.005244 | 1.26 |
| 221922_at | GPSM2 | 1p13.3 | 1.84 | 0.65 | 0.001804 | 1.32 |
| 220840_s_at | C1orf112 | 1q24.2 | 1.59 | 0.66 | 6.43 × 10⁻²⁵ | 6.24 |
| 202969_at | DYRK2 | 12q15 | 2.08 | 0.66 | 0.010667 | 1.12 |
| 218747_s_at | TAPBPL | 12p13.31 | 0.64 | 1.50 | 0.001252 | 0.69 |
| 211423_s_at | SC5DL | 11q23.3 | 0.50 | 1.54 | 0.002189 | 0.70 |
| 202109_at | ARFIP2 | 11p15 | 0.62 | 1.59 | 0.01092 | 0.83 |
| 210251_s_at | RUFY3 | 4q13.3 | 0.41 | 1.62 | 0.013938 | 0.82 |
| 201732_s_at | CLCN3 | 4q33 | 0.47 | 1.62 | 0.046582 | 0.82 |
| 206896_s_at | GNG7 | 19p13.3 | 0.58 | 1.74 | 0.000793 | 0.78 |
| 205992_s_at | IL15 | 4q31 | 0.23 | 1.82 | 0.004183 | 0.66 |
| 228003_at | RAB30 | 11q12-q14 | 0.49 | 2.09 | 0.004334 | 0.63 |
| 228582_x_at | — | — | 0.37 | 2.31 | 0.01386 | 0.95 |
| 201702_s_at | PPP1R10 | 6p21.3 | 0.28 | 2.31 | 0.001805 | 0.60 |
| 232323_s_at | TTC17 | 11p12-p11.2 | 0.48 | 3.01 | 0.000974 | 0.77 |

In conclusion, we have discovered that NEK2 over-expression relates to cell proliferation and drug resistance, resulting in early patient death in multiple primary cancers. Thus, targeting NEK2 by shRNA, siRNA, or a more specific kinase inhibitor to NEK2 or its signaling pathways represents a promising new strategy for the treatment of high risk myelomas or other cancers.

### Example 3

### Identification of Kinase Inhibitors to Target NEK2 in Cancer

By NEK2 kinase activity assay, we screened a Kinase Inhibitor Library from Calbiochem Inc. (San Diego, Calif.). This library contains 160 well-characterized, cell-permeable, potent, and reversible protein kinase inhibitors. We successfully identified 9 inhibitors that potently inhibit NEK2 activity at the dose of 5 μM (FIG. 7a). Further titration assays of these inhibitors, revealed that Inhibitor 2 (NI-2) had the lowest IC50 (67 nM). NI-2 is an inhibitor of serine-threonine kinase named KP372-1. We tested if the compound KP372-1 can inhibit NEK2 expression in cancer cell lines. As shown in FIG. 7b, KP372-1 significantly inhibits NEK2 expression, especially in NEK2 over-expressed in MM, lung cancer and breast cancer cells.

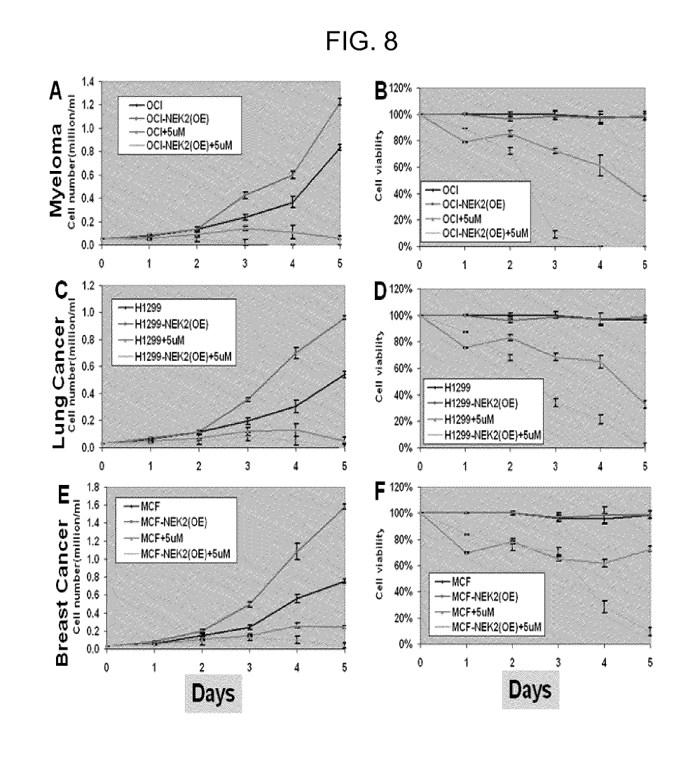

We then determined the cytotoxic effects of this compound KP372-1 on the NEK2-overexpressing myeloma cell lines. Cultures of cell lines transfected with a vector for overexpression of NEK2 (myeloma OCI-MY5, a lung cancer cell line H1299, and a breast cancer cell line MCF7) were treated with 5 μM KP372-1 for 5 days. Cell proliferation and cell death were evaluated. Empty vector (EV)-transfected cancer cells with or without treatment were used as controls. The kinase inhibitor KP372-1 induced significantly cell death and growth inhibition in both NEK2-transfected cancer cells and their EV control cells (FIG. 8). Furthermore, the KP372-1 showed stronger anti-proliferative and apoptotic activity in the NEK2-transfected cells than the EV control cells. These data indicate that KP372-1 is highly cytotoxic to NEK2-overexpressing myeloma and other cancer cells.

### Example 4

### Targeting NEK2 Can Overcome Drug Resistance

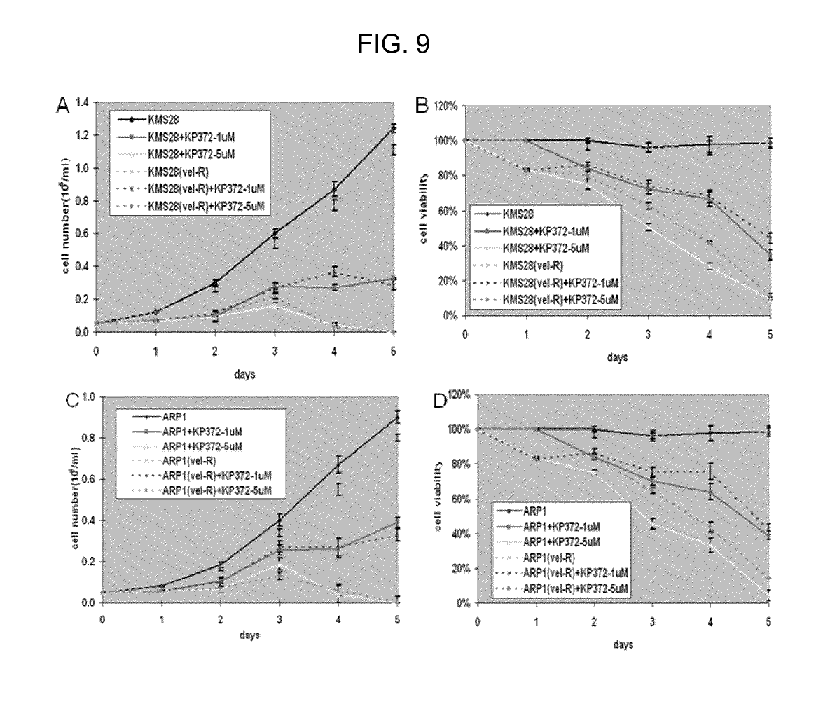

To determine whether the NEK2 inhibitor KP372-1 can overcome drug resistance in KMS28PE- and ARP1-bortezomib resistant myeloma cell lines, we treated with KP372-1 at concentrations of 1 μM and 5 μM. As shown in FIG. 9, KP372-1 treatment induced significant growth inhibition (FIGS. 9A & 9C) and cell death (FIGS. 9B & 9D) in both bortezomib-resistant (vel-R) and bortezomib-sensitive cell lines KMS28PE and ARP1; while bortezomib treatment induced significantly less growth inhibition and cell death in drug-resistant cells compared with their parental controls.

### Example 5

### KP372-1 Inhibits Multiple Myeloma Tumor Growth in a Mouse Model

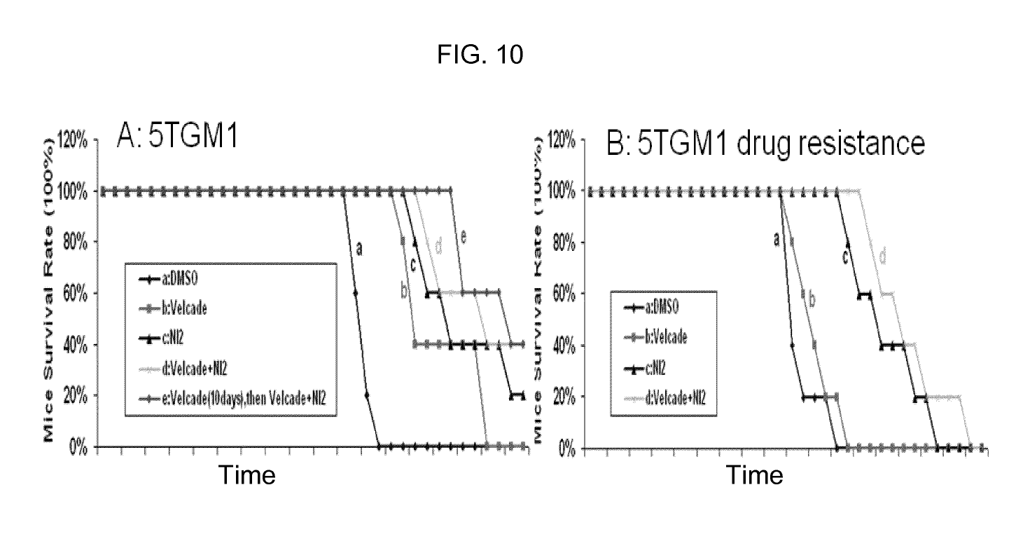

KP372-1 (NI-2) was used to treat 8 mice in each study group including four groups with single agent or combination with bortezomib (velcade). 1×10⁶ 5TGM1 multiple myeloma cells (in 100 μL PBS) were injected into each mouse through the tail vein and tumors were allowed to grow for 1 week. Treatment with bortezomib (1 mg/kg, 2 times/week, sc), NI-2 (0.8 mg/kg 3 times/week, sc), and the combination of KP372-1 (NI-2) with bortezomib (velcade) was performed 1 week after injection. As shown in FIG. 10A, KP372-1 treatment significantly inhibited the growth of 5TGM1 tumors (P<0.05), especially in the combination treatment with bortezomib, compared with untreated 5TGM1 control tumors.

We also developed a bortezomib-resistant 5TGM1 line (5TGM1-DR) using a stepwise increase of the bortezomib concentration in the cultures. The IC50s of bortezomib treatment in 5TGM1-R and wild-type 5TGM1 cells (5TGM1-WT) were 250.12 nM and 5.26 nM, respectively. We examined the effects of KP372-1 treatment on the growth of 5TGM1 myeloma mouse. KP372-1 was given to 8 mice in each of the five groups, including single agent or combination treatment with bortezomib: 5TGM1-WT, 5TGM1-DR, 5TGM1-DR+KP372-1 (0.8 mg/kg, 3 times/week, s.c), 5TGM1-DR+bortezomib (1 mg/kg, 2 times/week, s.c), 5TGM1-DR+bortezomib+KP372-1. As shown in FIGS. 10a & 10b, the untreated 5TGM1-DR mice had a significantly shorter survival compared with the wild-type 5TGMland KP372-1 was able to overcome drug resistance and extend mouse survival (P<0.01).

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for determining a diagnosis or prognosis of a neoplastic disease in a subject, the method comprising detecting the expression of one or more of NEK2, BIRC5, TOP2A, PHF19, EZH2, NUSAP1, NDC80. CDKN3, RHOC, and PTPRC in a test sample from the subject, wherein an elevation of one or more of NEK2, BIRC5, TOP2A, PHF19, EZH2, NUSAP1, NDC80. CDKN3, RHOC, and PTPRC expression in the subject compared to a reference level is an indication of a diagnosis of a neoplastic disease or an increased likelihood of recurrence of a neoplastic disease.

2. The method of claim 1, wherein an increase of NEK2, BIRC5, TOP2A, PHF19, EZH2, NUSAP1, NDC80. CDKN3, RHOC, and PTPRC expression in the subject compared to the reference level is an indication of drug-resistant multiple myeloma.

3. The method of claim 1, wherein an increase of NEK2 expression in the subject compared to the reference level is an indication of a drug-resistant cancer.

4. The method of claim 3, wherein the cancer is resistant to proteasome inhibitors.

5. The method of claim 4, wherein the proteasome inhibitor is selected from the group consisting of: bortezomib, Disulfiram, Salinosporamide A, Carfilzomib, CEP-18770 and MLN9708.

6. The method of claim 3, wherein the cancer is resistant to etoposide or doxorubicin.

7. The method of claim 1, wherein the neoplastic disease is multiple myeloma.

8. The method of claim 7, wherein an increase of NEK2 expression in the subject compared to the reference level is an indication of drug-resistant multiple myeloma.

9. The method of claim 1, wherein a difference in the level of NEK2 expression in the subject compared to a reference level is an indication of early multiple myeloma recurrence and decreased overall survival in a subject with multiple myeloma compared to a control subject that does not show a difference in the level of NEK2 expression.

10. The method of claim 9, wherein the difference in the level of NEK2 expression is an increase of NEK2 expression in the subject compared to the reference level.

11. The method of claim 1, wherein a difference in the level of NEK2 expression in the subject compared to a reference level is an indication of a diagnosis of small cell lung carcinoma, breast cancer, glioma, adult acute myeloid leukemia, bladder cancer, mantel cell lymphoma, or mesothelioma in the subject.

12. The method of claim 11, wherein the difference in the level of NEK2 expression is an increase of NEK2 expression in the subject compared to the reference level.

13. The method of claim 1, wherein the reference level is the level in a comparable sample from one or more healthy individuals.

14. The method of claim 1, wherein the detecting comprises amplifying a fragment of the NEK2, BIRC5, TOP2A, PHF19, EZH2, NUSAP1, NDC80. CDKN3, RHOC, or PTPRC mRNA.

15. The method of claim 14, wherein the amplifying is accomplished by polymerase chain reaction (PCR).

16. The method of claim 15 wherein the detecting comprises RT-PCR.

17. The method of claim 15, wherein the amplifying employs a detectably labeled primer or probe.

18. The method of claim 15, wherein the detecting is accomplished using real-time PCR.

19. The method of claim 1, wherein the detecting comprises measuring the presence, absence, or amount of a NEK2, BIRC5, TOP2A, PHF19, EZH2, NUSAP1, NDC80. CDKN3, RHOC, and PTPRC protein in the sample.

20. The method of claim 19, wherein the measuring uses an antibody that specifically binds to a NEK2, BIRC5, TOP2A, PHF19, EZH2, NUSAP1, NDC80. CDKN3, RHOC, and PTPRC protein.

21. The method of claim 20, wherein the measuring is by an ELISA assay, a Western blot assay, or an immunohistochemical assay.

22. The method of claim 1, wherein the sample is a body fluid sample or a biopsy sample.

23. The method of claim 1, wherein the subject is a human patient having or suspected of having a neoplastic disease.

24. The method of claim 1, wherein the subject is a human patient having or suspected of having multiple myeloma.
